(19) United States
(12) Patent Application Publication
(10) Pub. No.: US 2014/0228421 A1
(43) Pub. Date: Aug. 14, 2014

(54) Methods For Inhibiting Native And Promiscuous Uptake Of Monoamine Neurotransmitters

(71) Applicants: Anthony McKinney, Cambridge, MA (US); Frank Bymaster, Brownsburg, IN (US)

(72) Inventors: Anthony McKinney, Cambridge, MA (US); Frank Bymaster, Brownsburg, IN (US)

(21) Appl. No.: 13/907,948

(22) Filed: Jun. 2, 2013

Related U.S. Application Data
(63) Continuation of application No. 13/764,520, filed on Feb. 11, 2013, now abandoned.
Application No. 13/764,520 is a continuation of application No. 13/605,890, filed on Sep. 6, 2012, now abandoned.
(60) Provisional application No. 61/573,499, filed on Sep. 7, 2011.

(51) Int. Cl. C07D 209/52; A61K 45/06; A61K 31/198; A61K 31/403

(52) U.S. Cl. CPC C07D 209/52; A61K 31/403; A61K 45/06; A61K 31/198
USPC 514/412

(57) ABSTRACT

The present invention relates to methods of inhibiting native and promiscuous uptake of biogenic amine neurotransmitters with triple reuptake inhibitors in the treatment of conditions affected by monoamine neurotransmitters.

### RELATED APPLICATIONS

This application claims priority benefit of U.S. Provisional patent application Ser. No. 61/573,499, filed Sep. 7, 2011, the disclosure of which is incorporated herein in its entirety by reference.

### TECHNICAL FIELD

The present invention relates to use of compounds that inhibit native and promiscuous uptake of monoamine neurotransmitters to treat a central nervous system disorder.

### BACKGROUND OF THE INVENTION

Biogenic amines, including 5-hydroxytryptamine (serotonin), norepinephrine, and dopamine have been implicated in central nervous system disorders, including depression and other neuropsychiatric disorders, ranging from anxiety to eating disorders and drug addiction. Inhibitors that selectively inhibit the reuptake of these monoamine neurotransmitters have been shown to be efficacious in treating depression and other neuropsychiatric disorders. Currently approved pharmacotherapies for treating depression and other monoamine neurotransmitter-related conditions and disorders include compounds that inhibit one or two monoamine uptake transporters, such as those that inhibit serotonin or a combination of serotonin and norephinephrine. For example, selective serotonin reuptake inhibitors (SSRIs) and serotonin/norephinephrine reuptake inhibitors (SNRIs) and have widely been prescribed as anti-depressants.

However, the effectiveness of the SSRIs and SNRIs is limited since they have modest remission rates, do not treat some symptoms well, such as fatigue, anhedonia, and cognitive impairment, and have troublesome adverse events profiles (Trivedi et al. 2006; Papakostas, 2007). The limited effectiveness of these approved inhibitors might in part reflect promiscuous or heterologous uptake of the monoamine neurotransmitter. Under conditions resulting in high extracellular neurotransmitter levels, such as reuptake inhibition with a SSRI, extrasynaptic promiscuous uptake of one monoamine can occur by a non-native transporter (Daws, 2009). Examples of promiscuous uptake include dopamine uptake into norephinephrine neurons, dopamine uptake into serotonin neurons, and norepinephrine uptake into serotonin neurons (Carboni et al. 1990; Shen et al. 2004; Vizi et al. 2004). Promiscuous uptake may limit the extracellular level of the neurotransmitter, and consequently effectiveness, of the monoamine neurotransmitter that can be achieved by use of a SSRI, SNRI, or a similar single or double monoamine uptake inhibitor.

The serotonin transporter (SERT), norepinephrine transporter (NET), and the dopamine transporter (DAT) each have high affinity for their respective monoamine neurotransmitters. However, the transporters can promiscuously bind and uptake non-native neurotransmitters with a low affinity. The ability of monoamine transporters to promiscuously uptake non-native monoamine neurotransmitters has been demonstrated under conditions of high extracellular levels of monoamine neurotransmitters in studies with specific transporter inhibition or ablation of a transporter with gene knockout technologies (Daws, 2009).

While promiscuous uptake by monoamine transporters has been described, there has been little to no description of specific clinical applications involving promiscuous uptake. Furthermore, promiscuous uptake has not been a target for modulating biogenic amine activity or improving upon modulation of biogenic amine activity. Thus there remains a need for inhibiting promiscuous uptake in the treatment of conditions affected by monoamine neurotransmitters.

### SUMMARY OF EXEMPLARY EMBODIMENTS

Provided herein are methods using a triple reuptake inhibitor to inhibit native and promiscuous uptake of monoamine neurotransmitters for the treatment of humans suffering from signs and symptoms of central nervous system (CNS) disorders and other conditions amenable to treatment involving administration of a triple monoamine reuptake inhibitor. Such disorders and conditions include, but are not limited to, depression, treatment resistant depression, attention deficit hyperactivity disorder, an anxiety disorder, obesity, substance abuse, Parkinson's disease, chronic pain states such as neuropathic pain, fibromyalgia, traumatic brain injury, substance abuse, irritable bowel syndrome, and a cognitive disorder.

The methods provided herein utilize an effective triple reuptake inhibitor to inhibit both native and promiscuous uptake of monoamine neurotransmitters. These methods accordingly inhibit monoamine transporter native uptake of monoamine transmitters from the synapse, as well extrasynaptic transporter uptake of native monamine neurotransmitters that diffuse out of the synapse into extracellular space. Moreover, these methods inhibit monoamine transporter promiscuous uptake of extracellular non-native neurotransmitters. Promiscuous uptake of non-native neurotransmitters by monoamine transporters can occur under conditions where extracellular neurotransmitters levels rise to higher levels, to micromolar concentration ranges, as during transporter inhibition. Under such conditions, the neurotransmitter levels become high enough for heterologous uptake to occur, even though the non-native transporters have relatively low affinity for the non-native neurotransmitters (Daws, 2009). The methods of the present invention inhibit both native and promiscuous binding through use of a triple reuptake inhibitor, thereby allowing for greater extracellular levels of monoamine transmitters, and consequently greater therapeutic effects, than could be achieved by use of a single or dual reuptake inhibitor.

The methods of the present invention provide use of a triple reuptake inhibitor to inhibit both native and promiscuous uptake of monoamine neurotransmitters for the treatment of humans suffering from central nervous system disorders that may be alleviated by increasing extracellular levels of monoamine neurotransmitters. Such CNS disorders include, but are not limited to, depression disorders (for example, major depressive disorder, treatment resistant depression, and dysthymic disorder), cognitive disorders (such as Attention-Deficit/Hyperactivity Disorder, Predominately Inattentive Type; Attention-Deficit/Hyperactivity Disorder, Predominately Hyperactivity-Impulsive Type; Attention-Deficit/Hyperactivity Disorder, Combined Type; Conduct Disorder; Oppositional Defiant Disorder, mild cognitive impairment), as well as forms and symptoms of anxiety, alcohol abuse, drug abuse, obsessive compulsive behaviors, learning disorders, reading problems, gambling addiction, manic symptoms, phobias, panic attacks, academic problems in school, smoking, abnormal sexual behaviors, schizoid behaviors, somatization, sleep disorders, stuttering, tic disorders, Parkinson's disease, chronic pain states like neuropathic pain and fibromyalgia, and obesity.

Additionally provided herein are methods of treatment using a triple reuptake inhibitor to inhibit native and promiscuous uptake of monoamine neurotransmitters in combination and in coordination with an additional or secondary psychotherapeutic agent or drug. Suitable secondary psychotherapeutic drugs for use in the methods herein include, but are not limited to, drugs from the general classes of antipsychotic, antidepressants, anticonvulsant, anxiolytic, stimulant, antiaddictive, and appetite suppressants. (See, e.g., R J. Baldessarini in Goodman & Gilman's The Pharmacological Basis of Therapeutics, 11th Edition, Chapters 17 and 18, McGraw-Hill, 2005 for a review). Exemplary atypical antipsychotics include, for example, aripiprazole, ziprasidone, risperidone, quetiepine, or olanzapine. Exemplary antidepressants include, for example, tri-cyclic antidepressants (TCAs), specific monoamine reuptake inhibitors, selective serotonin reuptake inhibitors, selective norepinephrine or noradrenaline reuptake inhibitors, selective dopamine reuptake inhibitors, norepinephrine-dopamine reuptake inhibitors, serotonin-norepinephrine reuptake inhibitors, multiple monoamine reuptake inhibitors, monoamine oxidase inhibitors, and atypical antidepressants. Another suitable secondary drug would be levodopa (L-DOPA) for treatment of Parkinson's disease.

Additional background information pertaining to triple reuptake inhibitors useful in the methods of the present invention may be found, for example, in: U.S. Pat. No. 4,435,419, U.S. Pat. No. 6,372,919, U.S. Pat. No. 7,098,229, U.S. patent application Ser. No. 11/205,956, U.S. patent application Ser. No. 11/493,431, U.S. patent application Ser. No. 11/740,667, U.S. patent application Ser. No. 11/936,016, U.S. patent application Ser. No. 12/135,053, U.S. patent application Ser. No. 12/208,284, U.S. patent application Ser. No. 12/334,432, U.S. patent application Ser. No. 12/428,399, U.S. patent application Ser. No. 12/782,705, U.S. patent application Ser. No. 12/895,788, U.S. patent application Ser. No. 13/048,852, U.S. patent application Ser. No. 13/310,694, U.S. patent application Ser. No. 13/366,209, U.S. patent application Ser. No. 13/335,981, U.S. patent application Ser. No. 13/507,610, U.S. patent application Ser. No. 13/297,452, U.S. patent application Ser. No. 13/366,211, U.S. Provisional Application No. 61/662,462, U.S. Provisional Application No. 61/677,453, U.S. Provisional Application No. 61/573,499, U.S. Provisional Patent Application No. 61/682,314, U.S. Provisional Patent Application No. 61/682,315, and U.S. Provisional Patent Application No. 61/419,769, each of which is incorporated herein by reference in their entirety.

The present invention may be understood more fully by reference to the detailed description and examples which are intended to exemplify non-limiting embodiments of the invention.

### BRIEF DESCRIPTION OF THE DRAWINGS

FIG. 1 is a graph showing a decrease in patients' scores on the Montgomery Åsberg Depression Rating Scale when treated with EB-1010 ((+)-1-(3,4-dichlorophenyl)-3-azabicyclo[3.1.0]hexane) in comparison to placebo in a six week double-blind study and one week post-treatment (modified intent-to-treat, n=56) (mixed-effects model repeated measures approach (MMRM) least square (LS) means).

FIG. 2 is a graph showing that treatment with EB-1010 ((+)-1-(3,4-dichlorophenyl)-3-azabicyclo[3.1.0]hexane) resulted in a decrease on the Hamilton Depression Rating Scale (HAM-D) in comparison to placebo in a six week double-blind study and one week post-treatment (modified intent-to-treat, n=56) (MMRM LS means).

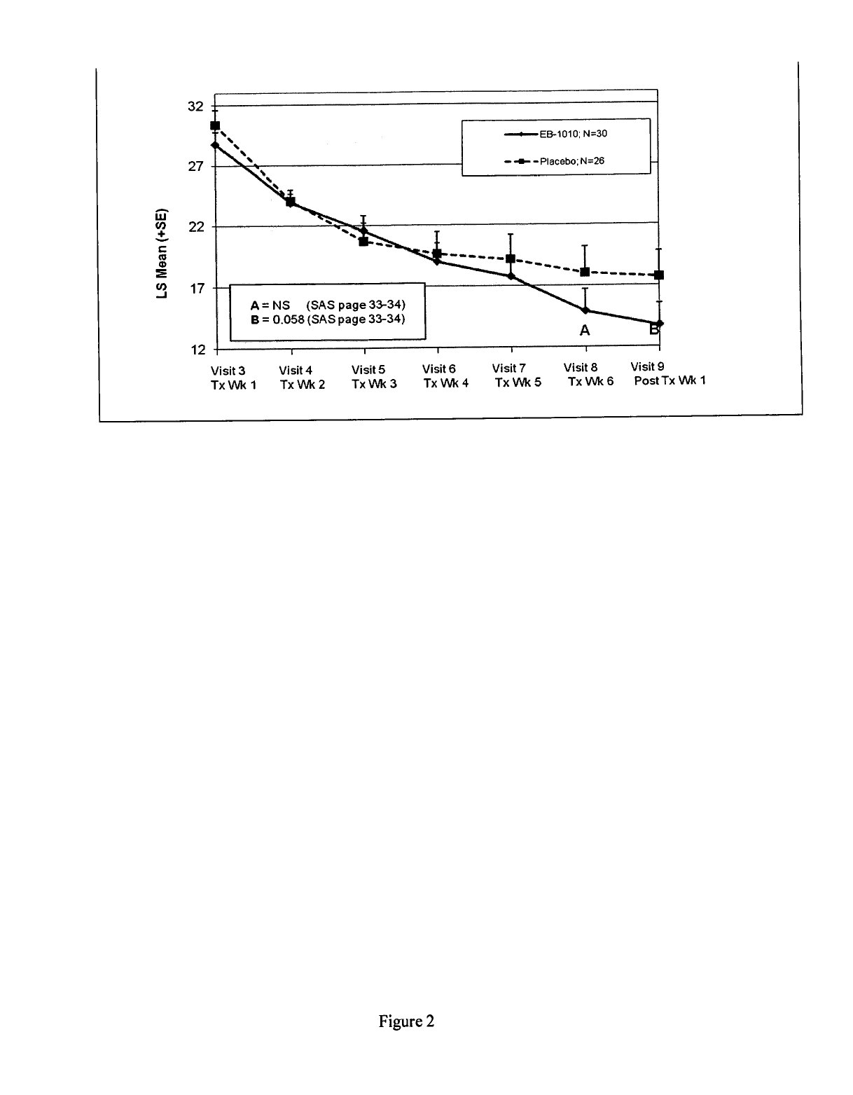

FIG. 3 is a graph showing that treatment with EB-1010 ((+)-1-(3,4-dichlorophenyl)-3-azabicyclo[3.1.0]hexane) resulted in a decrease on the Clinical Global Impression-Improvement Scale (CGI-I) in a six week double-blind study and one week post-treatment indicating improvement in the condition of the patients in a six week double-blind study and one week post-treatment (modified intent-to-treat, n=56) (MMRM LS means).

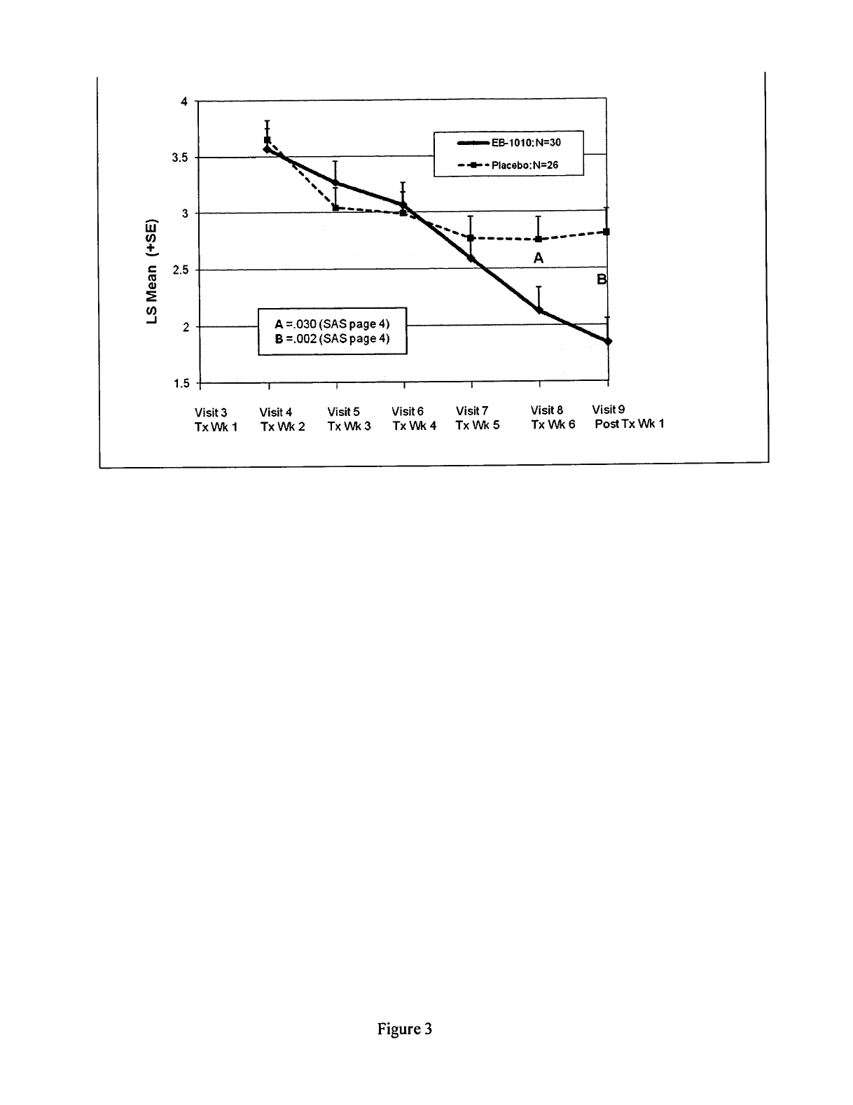

FIG. 4 is a graph showing an improvement in the condition of patients treated with EB-1010 ((+)-1-(3,4-dichlorophenyl)-3-azabicyclo[3.1.0]hexane) in comparison to placebo in a six week double-blind study and one week post-treatment as determined using the Clinical Global Impression-Severity (CGI-S) scale. (Modified intent-to-treat, n=56) (MMRM LS means).

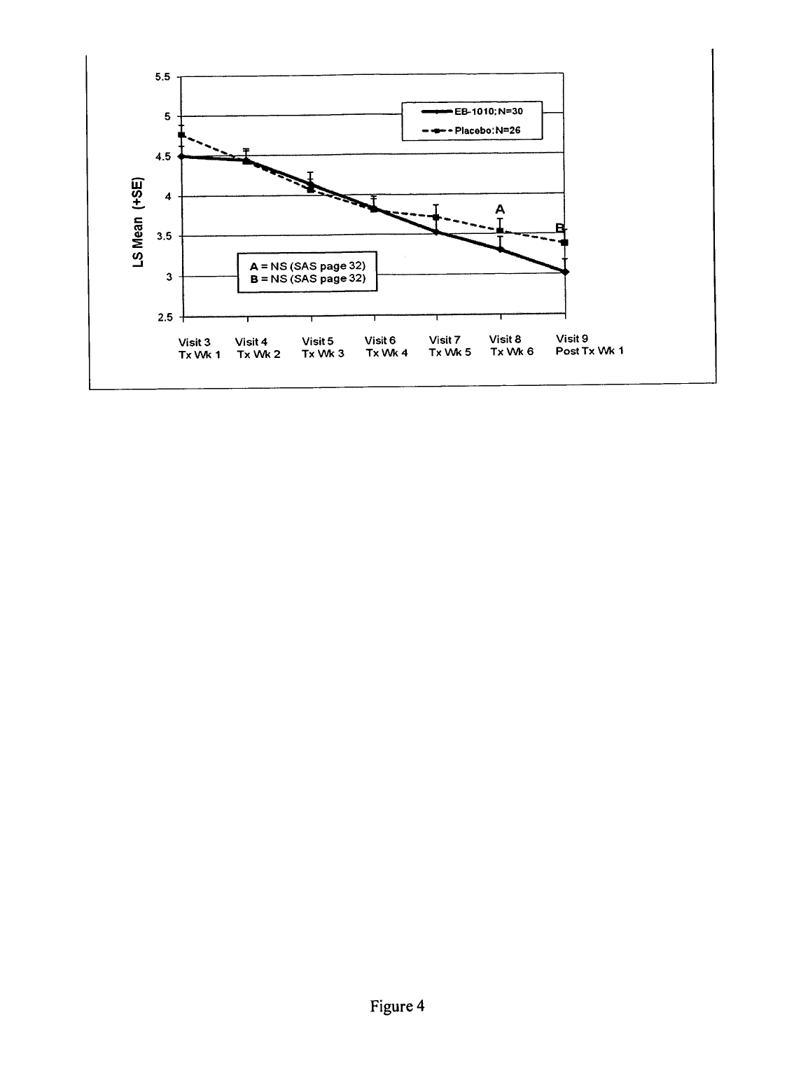

FIG. 5 is a graph showing that treatment with EB-1010 ((+)-1-(3,4-dichlorophenyl)-3-azabicyclo[3.1.0]hexane) resulted in significantly greater remission rates than treatment with placebo as measured by the Clinical Global Impressions-Severity (CGI-S) scale (Last Observation Carried Forward (LOCF)).

FIG. 6 is a graph showing that treatment with EB-1010 ((+)-1-(3,4-dichlorophenyl)-3-azabicyclo[3.1.0]hexane) resulted in statistically significant improvement on the adhedonia factor score of the MADRS compared to placebo in a six week double-blind study and one week post-treatment. (Modified intent-to-treat, n=56) (MMRM LS means).

FIG. 7 is a graph showing that Derogatis Interview for Sexual Functioning-Self Report (DISF-SR) scores stratified by low mean baseline scores versus high mean baseline scores and that there was no difference in those treated with EB-1010 ((+)-1-(3,4-dichlorophenyl)-3-azabicyclo[3.1.0]hexane) or placebo indicating that treatment with EB-1010 is not associated with the emergence of sexual dysfunction that is typically observed with serotonergic and serotonergic combination antidepressants (LOCF).

### DETAILED DESCRIPTION OF EXEMPLARY EMBODIMENTS

The present invention provides methods for treating or preventing a wide variety of disorders of the central nervous system (CNS), including neuropsychiatric disorders. Using the methods of the invention, CNS disorders are amenable to treatment, prophylaxis, and/or alleviation of the disorder and/or associated symptom(s) by inhibiting cellular native reuptake and promiscuous uptake of multiple biogenic amines causally linked to the targeted CNS disorder, wherein the biogenic amines targeted for uptake inhibition are norepinephrine, serotonin, and dopamine. The methods of the invention employ effective triple reuptake inhibitor compositions that inhibit cellular native and promiscuous uptake of norepinephrine, serotonin, and dopamine.

Monoaminergic neurotransmission largely occurs through wired, synaptic transmission but also occurs through extrasynaptic, volume transmission. The monoamine transporters that reuptake the monoamines norepinephrine, serotonin, and dopamine involved in wired, synaptic transmission are located perisynaptically on nerve terminals. There are also monoamine transporters located extrasynaptically along axonal membranes. Monoamine neurotransmitters that diffuse out of the synapse and into extracellular fluid may interact with native extrasynaptic receptors involved in volume or extrasynaptic neurotransmission, and the neurotransmitter may also be cleared by extrasynaptic native transporters (Fuxe et al. 2007; Zhou et al. 1998). Inhibition of monoamine uptake transporters has been shown to markedly increase the extracellular levels of the respective neurotransmitters levels above baseline, presumably due to diffusion of greater numbers of neurotransmitters out of the synapse (i.e. Bymaster et al. 2002a, 2002b). Under conditions of transporter inhibition, such as pharmacological inhibition or ablation of the transporter with gene knockout technologies, other transporters including non-native monoaminergic transporters, may promiscuously or heterologously uptake and clear the non-native neurotransmitter. Examples of promiscuous uptake include uptake of dopamine into norepinephrine neurons, dopamine uptake into serotonin neurons, and norepinephrine uptake into serotonin neurons (Carboni et al. 1990; Shen et al. 2004; Vizi et al. 2004). Promiscuous uptake occurs through the ability of monoamine transporters to bind non-native monoamines with a relatively low affinity. Native binding affinities are in the 50 to 100 nM range, while non-native binding affinities are in the uM range. For example, dopamine transporters have been estimated to bind serotonin with an affinity that is about 1/15 of the affinity for native dopamine (Zhou et al., 2005).

Inhibition of monoamine uptake transporters may therefore affect both native and promiscuous uptake of monoamine neurotransmitters. For example, a microdialysis study has shown that the combination of fluoxetine, a selective serotonin reuptake inhibitor, and bupropion, an inhibitor of norepinephrine and dopamine reuptake, produced greater increases in norepinephrine and dopamine in several brain regions than either inhibitor alone (Li et al. 2002). In another example, the selective norepinephrine transporter inhibitor atomoxetine increased extracellular norepinephrine and dopamine concentrations in prefrontal cortex where dopamine is cleared by the norepinephrine transporter (Bymaster et al. 2002a).

The present invention provides methods of treating or preventing a CNS disorder by administering a triple reuptake inhibitor that is sufficient to inhibit native and promiscuous uptake of norepinephrine, serotonin, and dopamine. The inhibition of three reuptake transporters and resultant blocking of promiscuous or heterologous uptake allows for greater extracellular levels of monoamine neurotransmitters than extracellular levels obtained by use of a single or dual reuptake inhibitor. The methods of the present invention therefore can increase the relative effects of each neurotransmitter, and thereby provide efficacy at lower doses than might be required with use of a single or dual reuptake inhibitor.

Overall, inhibiting native and promiscuous uptake of norepinephrine, serotonin, and dopamine will result in higher extracellular concentrations of all three monoamines, which may result in a greater effect with lower transporter occupancy. The lower transporter occupancy in turn could be obtained with a lower dose of reuptake inhibitor. For example, as shown herein, use of the triple reuptake inhibitor (+)-1-(3,4-dichlorophenyl)-3-azabicyclo[3.1.0]hexane was efficacious in treating depression, and may achieve efficacy at a relatively lower transporter occupancy than a SSRI. This triple reuptake inhibitor has an unbalanced serotonin-norepinephrine-dopamine in vitro reuptake inhibition ratio of ˜1:2:8, respectively (Skolnick et al., 2003). Example I provides human clinical trial evidence demonstrating the efficacy of (+)-1-(3,4-dichlorophenyl)-3-azabicyclo[3.1.0]hexane for treating depression. Example II provides human clinical trial evidence from an acute study that the level of serotonin transporter occupancy in the brain following administration of (+)-1-(3,4-dichlorophenyl)-3-azabicyclo[3.1.0]hexane, as determined by positron emission tomography, was approximately 48% and 32% at approximately 2 and 7 hours after dosing, respectively. In contrast, in a longer-term human clinical study of five SSRIs, serotonin transporter occupancy after four weeks was 80% at minimum therapeutic doses (Meyer et al., 2004). The lower transporter occupancies observed herein with a triple reuptake inhibitor are consistent with inhibition of promiscuous uptake resulting in higher levels of extracellular monoamine neurotransmitters.

As used herein, the terminology “triple reuptake inhibitor” refers to a compound that inhibits reuptake of norepinephrine, serotonin, and dopamine. The relative levels or ratios of reuptake inhibition may vary or be the same for each neurotransmitter, such that a triple reuptake inhibitor may be unbalanced or balanced with respect to its ability to uptake serotonin, norepinephrine, and dopamine.

The terms “promiscuous binding,” “promiscuous uptake,” “heterologous binding” and “heterologous uptake” are used interchangeably herein and refer to the ability of a monoamine transporter to bind and uptake a non-native monoamine neurotransmitter. Promiscuous binding/uptake therefore encompasses the serotonin transporter promiscuously binding/uptaking norepinephrine and dopamine; the norepinephrine transporter promiscuously binding/uptaking serotonin and dopamine; and the dopamine transporter binding/uptaking serotonin and norepinephrine.

The term “native” as used herein with respect to uptake or reuptake refers to a monoamine transporter binding and uptaking its native monoamine neurotransmitter, whereby the serotonin transporter binds/uptakes serotonin, the norepinephrine transporter binds/uptakes norepinephrine, and the dopamine transporter binds/uptakes dopamine.

The methods of the present invention utilize triple reuptake inhibitors that inhibit native and promiscuous reuptake of norepinephrine, serotonin, and dopamine. Exemplary triple reuptake inhibitor compounds include, but are not limited to, those described in U.S. patent application Ser. Nos. 12/334,432 and 12/895,788.

For example, the methods of the present invention includes use of compounds of the following formula I:

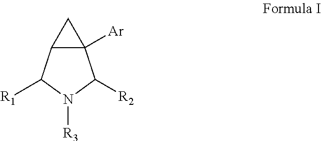

and enantiomers and pharmaceutically acceptable salts thereof, wherein:
Ar is a phenyl group substituted with two substituents independently selected from halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino;
R₁ and R₂ are independently selected from hydrogen, unsubstituted C₁₋₁₀ alkyl, C₃₋₁₀ alkenyl and C₃₋₁₀ alkynyl, and substituted C₁₋₁₀ alkyl, C₃₋₁₀ alkenyl and C₃₋₁₀ alkynyl wherein the substituent is one or more of hydroxy, cyano, halogen, C₁₋₆ alkoxy, aryl substituted C₁₋₆ alkoxy, aryloxy, aryloxy substituted with one or more halogens, C₁₋₆ alkyl, C₁₋₆ alkyl independently substituted with one or more of cyano and halogen, C₁₋₄ alkoxy, and C₁₋₄ haloalkoxy; and
R₃ is selected from hydrogen, C₁₋₆ alkyl, C₁₋₆ alkoxycarbonyl, C₂₋₆ alkanoyl, C₃₋₈ cycloalkyl, C₄₋₉ cycloalkanoyl, aryl, heteroaryl, saturated heterocyclic, C₂₋₁₀ alkenyl, C₂₋₁₀ alkynyl, and substituted C₁₋₆ alkyl, C₂₋₁₀ alkenyl and C₂₋₁₀ alkynyl wherein the substituent is one or more of cyano, halogen, hydroxy, C₁₋₆ alkoxy, C₁₋₆ alkoxycarbonyl, C₂₋₆ alkyloxycarbonyloxy, C₁₋₆ alkanoyl, C₁₋₆ alkanoyloxy, C₃₋₈ cycloalkyl, C₃₋₈ cycloalkyloxy, C₄₋₉ cycloalkanoyl, aryl, aryloxy, heteroaryl and saturated heterocyclic.

In certain embodiments, Ar is a phenyl group substituted with two substituents independently selected from methyl, ethyl, fluoro, chloro, trifluoromethyl, cyano, nitro, and trifluoromethoxy. In additional embodiments, R₁ and R₂ are hydrogen or methyl and R₃ is hydrogen, methyl, ethyl, propyl, isopropyl, butyl, isobutyl, tert-butyl or cyclopropyl.

Examples of compounds of formula I include, but are not limited to, 1-(2,4-difluorophenyl)-3-methyl-3-aza-bicyclo[3.1.0]hexane; 3-ethyl-1-(2,4-difluorophenyl)-3-aza-bicyclo[3.1.0]hexane; 1-(2,4-difluorophenyl)-3-isopropyl-3-aza-bicyclo[3.1.0]hexane; difluorophenyl)-3-aza-bicyclo[3.1.0]hexane; 1-(3,4-difluorophenyl)-3-methyl-3-aza-bicyclo[3.1.0]hexane; 1-(3,4-difluorophenyl)-3-ethyl-3-aza-bicyclo[3.1.0]hexane; (1R,5S)-3-ethyl-1-(3,4-difluorophenyl)-3-aza-bicyclo[3.1.0]hexane; (1S,5R)-3-ethyl-1-(3,4-difluorophenyl)-3-aza-bicyclo[3.1.0]hexane; 1-(3,4-difluorophenyl)-3-isopropyl-3-aza-bicyclo[3.1.0]hexane; 1-(3-chloro-4-fluorophenyl)-3-aza-bicyclo[3.1.0]hexane; (1R,5S)-1-(3-chloro-4-fluorophenyl)-3-aza-bicyclo[3.1.0]hexane; (1S,5R)-1-(3-chloro-4-fluorophenyl)-3-aza-bicyclo[3.1.0]hexane; 1-(3-chloro-4-fluorophenyl)-3-methyl-3-aza-bicyclo[3.1.0]hexane; (1R,5S)-1-(3-chloro-4-fluorophenyl)-3-methyl-3-aza-bicyclo[3.1.0]hexane; (1S,5R)-1-(3-chloro-4-fluorophenyl)-3-methyl-3-aza-bicyclo[3.1.0]hexane; 1-(3-chloro-4-fluorophenyl)-3-ethyl-3-aza-bicyclo[3.1.0]hexane; (1R,5S)-1-(3-chloro-4-fluorophenyl)-3-ethyl-3-aza-bicyclo[3.1.0]hexane; (1S,5R)-1-(3-chloro-4-fluorophenyl)-3-ethyl-3-aza-bicyclo[3.1.0]hexane; 1-(3-chloro-4-fluorophenyl)-3-isopropyl-3-aza-bicyclo[3.1.0]hexane; (1R,5S)-1-(3-chloro-4-fluorophenyl)-3-isopropyl-3-aza-bicyclo[3.1.0]hexane; (1S,5R)-1-(3-chloro-4-fluorophenyl)-3-isopropyl-3-aza-bicyclo[3.1.0]hexane; (1R,5S)-1-(4-chloro-3-fluorophenyl)-3-aza-bicyclo[3.1.0]hexane; (1S,5R)-1-(4-chloro-3-fluorophenyl)-3-aza-bicyclo[3.1.0]hexane; (1R,5S)-1-(4-chloro-3-fluorophenyl)-3-methyl-3-aza-bicyclo[3.1.0]hexane; (1S,5R)-1-(4-chloro-3-fluorophenyl)-3-methyl-3-aza-bicyclo[3.1.0]hexane; 1-(2,4-dichlorophenyl)-3-methyl-3-aza-bicyclo[3.1.0]hexane; dichlorophenyl)-3-ethyl-3-aza-bicyclo[3.1.0]hexane; 1-(2,4-dichlorophenyl)-3-isopropyl-3-aza-bicyclo[3.1.0]hexane; 1-(4-fluoro-3-methylphenyl)-3-aza-bicyclo[3.1.0]hexane; 1-(4-fluoro-3-methylphenyl)-3-methyl-3-aza-bicyclo[3.1.0]hexane; 3-ethyl-1-(4-fluoro-3-methylphenyl)-3-aza-bicyclo[3.1.0]hexane; 1-(4-fluoro-3-methylphenyl)-3-isopropyl-3-aza-bicyclo[3.1.0]hexane; 1-(3-fluoro-4-methylphenyl)-3-aza-bicyclo[3.1.0]hexane; (1R,5S)-1-(3-fluoro-4-methylphenyl)-3-aza-bicyclo[3.1.0]hexane; (1S,5R)-1-(3-fluoro-4-methylphenyl)-3-aza-bicyclo[3.1.0]hexane; 1-(3-fluoro-4-methylphenyl)-3-methyl-3-aza-bicyclo[3.1.0]hexane; (1R,5S)-1-(3-fluoro-4-methylphenyl)-3-methyl-3-aza-bicyclo[3.1.0]hexane; (1S,5R)-1-(3-fluoro-4-methylphenyl)-3-methyl-3-aza-bicyclo[3.1.0]hexane; 1-(3-fluoro-4-methylphenyl)-3-ethyl-3-aza-bicyclo[3.1.0]hexane; 1-(3-fluoro-4-methylphenyl)-3-isopropyl-3-aza-bicyclo[3.1.0]hexane; 1-(3-fluoro-4-methoxyphenyl)-3-aza-bicyclo[3.1.0]hexane; 1-(3-fluoro-4-(trifluoromethoxy)phenyl)-3-aza-bicyclo[3.1.0]hexane; (1R,5S)-1-(4-chloro-3-(trifluoromethyl)phenyl)-3-aza-bicyclo[3.1.0]hexane; (1S,5R)-1-(4-chloro-3-(trifluoromethyl)phenyl)-3-aza-bicyclo[3.1.0]hexane; (1R,5S)-1-(4-chloro-3-(trifluoromethyl)phenyl)-3-methyl-3-aza-bicyclo[3.1.0]hexane; (1S,5R)-1-(4-chloro-3-(trifluoromethyl)phenyl)-3-methyl-3-aza-bicyclo[3.1.0]hexane; and 1-(3-chloro-4-nitrophenyl)-3-methyl-3-aza-bicyclo[3.1.0]hexane. Cis-1-(3,4-dichlorophenyl)-2-methyl-3-aza-bicyclo[3.1.0]hexane; Cis-1-(3,4-dichlorophenyl)-2,3-dimethyl-3-aza-bicyclo[3.1.0]hexane; Trans-1-(3,4-dichlorophenyl)-2-methyl-3-aza-bicyclo[3.1.0]hexane; Trans-1-(3,4-dichlorophenyl)-2,3-dimethyl-3-aza-bicyclo[3.1.0]hexane; Cis-1-(3,4-dichlorophenyl)-4-methyl-3-aza-bicyclo[3.1.0]hexane; Trans-1-(3,4-dichlorophenyl)-4-methyl-3-aza-bicyclo[3.1.0]hexane; Trans-1-(3,4-dichlorophenyl)-3,4-dimethyl-3-aza-bicyclo[3.1.0]hexane; (1R,5S)-1-(3,4-dichlorophenyl)-3-methyl-3-aza-bicyclo[3.1.0]hexane; (1S,5R)-1-(3,4-dichlorophenyl)-3-methyl-3-aza-bicyclo[3.1.0]hexane; (1R,5S)-1-(3,4-dichlorophenyl)-3-ethyl-3-aza-bicyclo[3.1.0]hexane; (1S,5R)-1-(3,4-dichlorophenyl)-3-ethyl-3-aza-bicyclo[3.1.0]hexane; 1-(3,4-dichlorophenyl)-3-propyl-3-aza-bicyclo[3.1.0]hexane; (1R,5S)-1-(3,4-dichlorophenyl)-3-propyl-3-aza-bicyclo[3.1.0]hexane; (1S,5R)-1-(3,4-dichlorophenyl)-3-propyl-3-aza-bicyclo[3.1.0]hexane; 1-(3,4-dichlorophenyl)-3-isopropyl-3-aza-bicyclo[3.1.0]hexane; (1R,5S)-1-(3,4-dichlorophenyl)-3-isopropyl-3-aza-bicyclo[3.1.0]hexane; (1S,5R)-1-(3,4-dichlorophenyl)-3-isopropyl-3-aza-bicyclo[3.1.0]hexane; 1-(3,4-dichlorophenyl)-3-cyclopropyl-3-aza-bicyclo[3.1.0]hexane;

Additional examples of compounds of formula I include, but are not limited to (1R,5S)-1-(3,4-dichlorophenyl)-3-cyclopropyl-3-aza-bicyclo[3.1.0]hexane; (1S,5R)-1-(3,4-dichlorophenyl)-3-cyclopropyl-3-aza-bicyclo[3.1.0]hexane; 3-butyl-1-(3,4-dichlorophenyl)-3-aza-bicyclo[3.1.0]hexane; (1R,5S)-3-butyl-1-(3,4-dichlorophenyl)-3-aza-bicyclo[3.1.0]hexane; (1S,5R)-3-butyl-1-(3,4-dichlorophenyl)-3-aza-bicyclo[3.1.0]hexane; 1-(3,4-dichlorophenyl)-3-isobutyl-3-aza-bicyclo[3.1.0]hexane; (1R,5S)-1-(3,4-dichlorophenyl)-3-isobutyl-3-aza-bicyclo[3.1.0]hexane; (1S,5R)-1-(3,4-dichlorophenyl)-3-isobutyl-3-aza-bicyclo[3.1.0]hexane; 3-tert-butyl-1-(3,4-dichlorophenyl)-3-aza-bicyclo[3.1.0]hexane; (1R,5S)-3-tert-butyl-1-(3,4-dichlorophenyl)-3-aza-bicyclo[3.1.0]hexane; and (1S,5R)-3-tert-butyl-1-(3,4-dichlorophenyl)-3-aza-bicyclo[3.1.0]hexane.

In a preferred embodiment, the methods of the present invention utilize the compound (+)-1-(3,4-dichlorophenyl)-3-azabicyclo[3.1.0]hexane. (+)-1-(3,4-dichlorophenyl)-3-azabicyclo[3.1.0]hexane exists in at least three polymorphic forms, polymorphs A, B and C, as described in U.S. Provisional Patent Application No. 61/419,769, which incorporated herein by reference in its entirety. The polymorphs may be used in pharmaceutical compositions in combination or in forms that are substantially free of one or more of the other polymorphic forms. Synthesis of (+)-1-(3,4-dichlorophenyl)-3-azabicyclo[3.1.0]hexane and its polymorphs A, B and C may be carried out by methods described in U.S. Provisional Patent Application No. 61/419,769.

In another example, the methods of the present invention includes use of compounds of the following formula II:

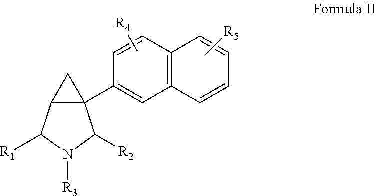

and enantiomers and pharmaceutically acceptable salts thereof, wherein:
R₁ and R₂ are independently selected from hydrogen, unsubstituted C₁₋₁₀ alkyl, C₃₋₁₀ alkenyl and C₃₋₁₀ alkynyl, and substituted C₁₋₁₀ alkyl, C₃₋₁₀ alkenyl and C₃₋₁₀ alkynyl wherein the substituent is one or more of hydroxy, cyano, halogen, C₁₋₆ alkoxy, aryl substituted C₁₋₆ alkoxy, aryloxy, aryloxy substituted with one or more halogens, C₁₋₆ alkyl, C₁₋₆ alkyl independently substituted with one or more of cyano and halogen, C₁₋₄ alkoxy, and C₁₋₄ haloalkoxy;
R₃ is selected from hydrogen, C₁₋₆ alkyl, C₁₋₆ alkoxycarbonyl, C₂₋₆ alkanoyl, C₃₋₈ cycloalkyl, C₄₋₉ cycloalkanoyl, aryl, heteroaryl, saturated heterocyclic, C₂₋₁₀ alkenyl, C₂₋₁₀ alkynyl, and substituted C₁₋₆ alkyl, C₂₋₁₀ alkenyl and C₂₋₁₀ alkynyl wherein the substituent is one or more of cyano, halogen, hydroxy, C₁₋₆ alkoxy, C₁₋₆ alkoxycarbonyl, C₂₋₆ alkyloxycarbonyloxy, C₁₋₆ alkanoyl, C₁₋₆ alkanoyloxy, C₃₋₈ cycloalkyl, C₃₋₈ cycloalkyloxy, C₄₋₉ cycloalkanoyl, aryl, aryloxy, heteroaryl and saturated heterocyclic; and
R₄ and R₅ are independently hydrogen or 1-4 substituents independently selected from halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.

Exemplary compounds of formula II include, but are not limited to, 1-(naphthalen-2-yl)-3-aza-bicyclo[3.1.0]hexane; (1R,5S)-1-(naphthalen-2-yl)-3-aza-bicyclo[3.1.0]hexane; (1S,5R)-1-(naphthalen-2-yl)-3-aza-bicyclo[3.1.0]hexane; 3-methyl-1-(naphthalen-2-yl)-3-aza-bicyclo[3.1.0]hexane; (1R,5S)-3-methyl-1-(naphthalen-2-yl)-3-aza-bicyclo[3.1.0]hexane; (1S,5R)-3-methyl-1-(naphthalen-2-yl)-3-aza-bicyclo[3.1.0]hexane; 3-ethyl-1-(naphthalen-2-yl)-3-aza-bicyclo[3.1.0]hexane; 3-isopropyl-1-(naphthalen-2-yl)-3-aza-bicyclo[3.1.0]hexane; (1R,5S)-3-isopropyl-1-(naphthalen-2-yl)-3-aza-bicyclo[3.1.0]hexane; (1S,5R)-3-isopropyl-1-(naphthalen-2-yl)-3-aza-bicyclo[3.1.0]hexane; 1-(2-methoxynaphthalen-6-yl)-3-aza-bicyclo[3.1.0]hexane; 1-(2-methoxynaphthalen-6-yl)-3-methyl-3-aza-bicyclo[3.1.0]hexane; 1-(2-ethoxynaphthalen-6-yl)-3-aza-bicyclo[3.1.0]hexane; and 1-(2-ethoxynaphthalen-6-yl)-3-methyl-3-aza-bicyclo[3.1.0]hexane.

In a preferred embodiment, the methods of the present invention utilize the compound (1R,5S)-(+)-1-(naphthalen-2-yl)-3-azabicyclo[3.1.0]hexane.

In a further example, the methods of the present invention includes use of compounds of the following formula III:

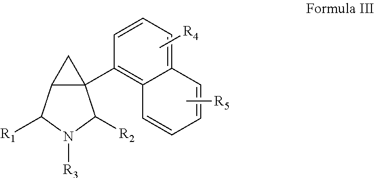

and enantiomers and pharmaceutically acceptable salts thereof, wherein:
R₁ and R₂ are independently selected from hydrogen, unsubstituted C₁₋₁₀ alkyl, C₃₋₁₀ alkenyl and C₃₋₁₀ alkynyl, and substituted C₁₋₁₀ alkyl, C₃₋₁₀ alkenyl and C₃₋₁₀ alkynyl wherein the substituent is one or more of hydroxy, cyano, halogen, C₁₋₆ alkoxy, aryl substituted C₁₋₆ alkoxy, aryloxy, aryloxy substituted with one or more halogens, C₁₋₆ alkyl, C₁₋₆ alkyl independently substituted with one or more of cyano and halogen, C₁₋₄ alkoxy, and C₁₋₄ haloalkoxy;
R₃ is selected from hydrogen, C₁₋₆ alkyl, C₁₋₆ alkoxycarbonyl, C₂₋₆ alkanoyl, C₃₋₈ cycloalkyl, C₄₋₉ cycloalkanoyl, aryl, heteroaryl, saturated heterocyclic, C₂₋₁₀ alkenyl, C₂₋₁₀ alkynyl, and substituted C₁₋₆ alkyl, C₂₋₁₀ alkenyl and C₂₋₁₀ alkynyl wherein the substituent is one or more of cyano, halogen, hydroxy, C₁₋₆ alkoxy, C₁₋₆ alkoxycarbonyl, C₂₋₆ alkyloxycarbonyloxy, C₁₋₆ alkanoyl, C₁₋₆ alkanoyloxy, C₃₋₈ cycloalkyl, C₃₋₈ cycloalkyloxy, C₄₋₉ cycloalkanoyl, aryl, aryloxy, heteroaryl and saturated heterocyclic; and
R₄ and R₅ are independently hydrogen or 1-4 substituents independently selected from halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.

Examples of formula III compounds include, but are not limited to, 1-(naphthalen-1-yl)-3-aza-bicyclo[3.1.0]hexane; (1R,5S)-1-(naphthalen-1-yl)-3-aza-bicyclo[3.1.0]hexane; (1S,5R)-1-(naphthalen-1-yl)-3-aza-bicyclo[3.1.0]hexane; 3-methyl-1-(naphthalen-1-yl)-3-aza-bicyclo[3.1.0]hexane; (1R,5S)-3-methyl-1-(naphthalen-1-yl)-3-aza-bicyclo[3.1.0]hexane; (1S,5R)-3-methyl-1-(naphthalen-1-yl)-3-aza-bicyclo[3.1.0]hexane; 1-(1-fluoronaphthalen-4-yl)-3-aza-bicyclo[3.1.0]hexane; 1-(1-fluoronaphthalen-4-yl)-3-methyl-3-aza-bicyclo[3.1.0]hexane; 1-(1-methylnaphthalen-4-yl)-3-aza-bicyclo[3.1.0]hexane; and 3-methyl-1-(1-methylnaphthalen-4-yl)-3-aza-bicyclo[3.1.0]hexane.

It will be understood that the exemplary, multiply aryl-substituted compounds identified above are illustrative, and that the subject modifications comprising multiple aryl substitutions can be varied to comprise other substituents, can include yet additional substituents (e.g., three or more substitutions on the aryl ring), combined with one another, or additionally combined with one or more substitutions on the azabicyclo[3.1.0]hexane ring, to yield yet additional compounds useful within the invention for treating CNS disorders (including a range of neuropsychiatric disorders, such as depression and anxiety).

In yet another example, the methods of the present invention includes use of compounds of the following formula IV:

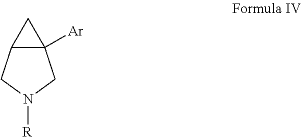

wherein Ar is a heterocyclic aryl group, optionally with or without substitution groups on the aryl ring, and wherein R is H or an optional substituent selected from, for example, hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl and cyano(C₁₋₆)alkyl, more preferably, methyl, ethyl, trifluoromethyl, trifluoroethyl and 2-methoxyethyl.

In an additional example, the methods of the present invention includes use of compounds of the following formula V:

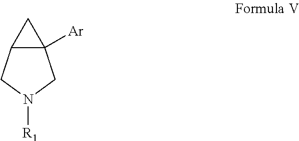

and enantiomers and pharmaceutically acceptable salts thereof, wherein:
Ar is a heterocyclic aryl group selected from furan, thiophene, pyrrole, imidazole, pyrazole, triazole, tetrazole, thiazole, isothiazole, pyridine, pyridizine, pyrimidine, pyrazine, triazine, indole, benzofuran, benzothiophene, benzothiazole, quinoline, isoquinoline, cinnoline, phthalazine, quinazoline, quinoxaline, chromane and isochromane, and Ar is either unsubstituted or substituted with one or more substituents independently selected from fluoro, chloro, bromo, iodo, —NO₂, —CN, —NH₂, carboxy, C₁₋₈ alkyl, C₂₋₈ alkenyl, C₂₋₈ alkynyl, halo(C₁₋₈)alkyl, hydroxy, trifluoromethyl, C₃₋₈ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxyl, C₁₋₈ alkylamino, and di(C₁₋₈)alkylamino; and
R₁ is selected from hydrogen, unsubstituted C₁₋₁₀ alkyl, C₃₋₈ cycloalkyl, C₂₋₁₀ alkenyl, and C₃₋₁₀ alkynyl, and substituted C₁₋₁₀ alkyl, C₃₋₁₀ alkenyl and C₃₋₁₀ alkynyl wherein the substituent is one or more of hydroxy, cyano, halogen, C₁₋₆ alkoxy, aryl substituted C₁₋₆ alkoxy, aryloxy, aryloxy substituted with one or more halogens, C₁₋₆ alkyl, C₁₋₆ alkyl independently substituted with one or more of cyano and halogen, C₁₋₄ alkoxy, and C₁₋₄ haloalkoxy.

Examples of formula V compounds include, but are not limited to, 1-(5-methylfuran-2-yl)-3-azabicyclo[3.1.0]hexane; 3-methyl-1-(5-methylthiophen-2-yl)-3-azabicyclo[3.1.0]hexane; 1-(5-methylthiophen-2-yl)-3-azabicyclo[3.1.0]hexane; 1-(pyridin-2-yl)-3-azabicyclo[3.1.0]hexane; 3-methyl-1-(pyridin-2-yl)-3-azabicyclo[3.1.0]hexane; 1-(pyridin-3-yl)-3-azabicyclo[3.1.0]hexane; 3-methyl-1-(pyridin-3-yl)-3-azabicyclo[3.1.0]hexane; 1-(pyridin-4-yl)-3-azabicyclo[3.1.0]hexane; 3-methyl-1-(pyridin-4-yl)-3-azabicyclo[3.1.0]hexane; 1-(6-methoxypyridin-3-yl)-3-azabicyclo[3.1.0]hexane; 1-(6-methoxypyridin-3-yl)-3-methyl-3-azabicyclo[3.1.0]hexane; 1-(benzofuran-2-yl)-3-methyl-3-azabicyclo[3.1.0]hexane; 1-(benzofuran-3-yl)-3-azabicyclo[3.1.0]hexane; 1-(benzofuran-3-yl)-3-methyl-3-azabicyclo[3.1.0]hexane; 1-methyl-2-(3-methyl-3-azabicyclo[3.1.0]hexan-1-yl)-1H-indole; 2-(3-ethyl-3-azabicyclo[3.1.0]hexan-1-yl)-1-methyl-1H-indole; 2-(3-isopropyl-3-azabicyclo[3.1.0]hexan-1-yl)-1-methyl-1H-indole; 1-methyl-5-(3-methyl-3-azabicyclo[3.1.0]hexan-1-yl)-1H-indole; 5-(3-ethyl-3-azabicyclo[3.1.0]hexan-1-yl)-1-methyl-1H-indole; 5-(3-isopropyl-3-azabicyclo[3.1.0]hexan-1-yl)-1-methyl-1H-indole; (1S,5S)-1-(benzo[b]thiophen-2-yl)-3-azabicyclo[3.1.0]hexane; (1R,5R)-1-(benzo[b]thiophen-2-yl)-3-azabicyclo[3.1.0]hexane; (1S,5S)-1-(benzo[b]thiophen-2-yl)-3-methyl-3-azabicyclo[3.1.0]hexane; (1R,5R)-1-(benzo[b]thiophen-2-yl)-3-methyl-azabicyclo[3.1.0]hexane; 1-(5-chlorobenzo[b]thiophen-3-yl)-3-azabicyclo[3.1.0]hexane; 1-(5-chlorobenzo[b]thiophen-2-yl)-3-methyl-3-azabicyclo[3.1.0]hexane; 1-(5-chlorobenzo[b]thiophen-3-yl)-3-methyl-3-azabicyclo[3.1.0]hexane; 1-(benzo[b]thiophen-2-yl)-3-aza-bicyclo[3.1.0]hexane; 1-(benzo[b]thiophen-2-yl)-3-methyl-3-aza-bicyclo[3.1.0]hexane; 1-(6-fluorobenzo[b]thiophen-2-yl)-3-aza-bicyclo[3.1.0]hexane; 1-(6-fluorobenzo[b]thiophen-2-yl)-3-methyl-3-aza-bicyclo[3.1.0]hexane; 1-(5-fluorobenzo[b]thiophen-2-yl)-3-aza-bicyclo[3.1.0]hexane; 1-(5-fluorobenzo[b]thiophen-2-yl)-3-methyl-3-aza-bicyclo[3.1.0]hexane, 2-(3-azabicyclo[3.1.0]hexan-1-yl)-benzo[d]thiazole, 1-(5-fluorobenzo[b]thiophen-2-yl)-3-aza-bicyclo[3.1.0]hexane, 1-(6-fluorobenzo[b]thiophen-2-yl)-3-aza-bicyclo[3.1.0]hexane, (1S)-1-(6-fluorobenzo[b]thiophen-2-yl)-3-azabicyclo[3.1.0]hexane, (1R)-1-(6-fluorobenzo[b]thiophen-2-yl)-3-azabicyclo[3.1.0]hexane; 5-(3-azabicyclo[3.1.0]hexan-1-yl)quinoline; 5-(3-methyl-3-azabicyclo[3.1.0]hexan-1-yl)quinoline; 5-(3-ethyl-3-azabicyclo[3.1.0]hexan-1-yl)quinoline; 5-(3-isopropyl-3-azabicyclo[3.1.0]hexan-1-yl)quinoline; 3-(3-azabicyclo[3.1.0]hexan-1-yl)quinoline; 3-(3-methyl-3-azabicyclo[3.1.0]hexan-1-yl)quinoline; 3-(3-ethyl-3-azabicyclo[3.1.0]hexan-1-yl)quinoline; 3-(3-isopropyl-3-azabicyclo[3.1.0]hexan-1-yl)quinoline; 6-(3-azabicyclo[3.1.0]hexan-1-yl)quinoline; 6-(3-methyl-3-azabicyclo[3.1.0]hexan-1-yl)quinoline; 6-(3-ethyl-3-aza-bicyclo[3.1.0]hexan-1-yl)quinoline; and 6-(3-isopropyl-3-azabicyclo[3.1.0]hexan-1-yl)quinolone.

It will be understood that the exemplary compounds identified above are illustrative, and that the heteroaryl ring can be varied to comprise other substituents, and/or can include yet additional substituents (i.e., three or more substitutions on the heteroaryl ring), combined with one another, or additionally combined with or without substitutions on the nitrogen atom as described herein, to yield yet additional compounds useful within the invention for treating CNS disorders (including a range of neuropsychiatric disorders, such as depression and anxiety).

The present invention provides methods for treating CNS disorders, including but not limited to, neuropsychiatric conditions, such as depression and anxiety. Suitable forms of triple reuptake inhibitors for the methods of the invention include the compounds exemplified herein, as well as their pharmaceutically acceptable salts, polymorphs, solvates, hydrates, and/or prodrugs, or any combination thereof.

The compounds described herein may be prepared using methods known to those skilled in the art. Compounds of formulas I, II and III may be synthesized according to methods described in U.S. patent application Ser. Nos. 11/493,431 and 12/334,432, each of which is incorporated herein by reference in their entirety. Compounds of formulas IV and V may be synthesized according to methods described in U.S. patent application Ser. Nos. 12/135,053 and 12/895,788, each of which is incorporated herein by reference in their entirety.

As indicated above, the compounds described herein encompass enantiomeric forms having chiral symmetric structure, which provide yet additional drug candidates for treating CNS disorders. Provided herein are enantiomers, diastereomers, and other stereoisomeric forms of the disclosed compounds, including racemic and resolved forms and mixtures thereof. The individual enantiomers may be separated according to methods that are well known to those of ordinary skill in the art. In certain embodiments, the enantiomers, diastereomers and other stereoisomeric forms of the disclosed compounds are substantially free of the corresponding enantiomers, diastereomers and stereoisomers. In other embodiments, the enantiomers, diastereomers and other stereoisomeric forms of the disclosed compounds contain no more than about 10%, about 5%, about 2% or about 1% of the corresponding enantiomers, diastereomers and stereoisomers. When the compounds described herein contain olefinic double bonds or other centers of geometric asymmetry, and unless specified otherwise, it is intended to include both E and Z geometric isomers. All tautomers are intended to be encompassed by the present invention as well

The triple reuptake inhibitor compounds described herein can be prepared as both acid addition salts formed from an acid and the basic nitrogen group of 1-aryl-3-azabicyclo[3.1.0]hexanes and base salts. Suitable acid addition salts are formed from acids which form non-toxic salts and include, for example, hydrochloride, hydrobromide, hydroiodide, sulphate, hydrogen sulphate, nitrate, phosphate, and hydrogen phosphate salts. Other examples of pharmaceutically acceptable addition salts include inorganic and organic acid addition salts. Additional pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. Suitable base salts are formed from bases which form non-toxic salts and include, for example, aluminum, calcium, lithium, magnesium, potassium, sodium, zinc and diethanolamine salts.

Prodrugs of the disclosed compounds may also be used in the present invention. Prodrugs are considered to be any covalently bonded carriers which release the active parent drug in vivo. Examples of prodrugs include esters or amides of a compound of the present invention with hydroxyalkyl or aminoalkyl as a substituent. These may be prepared by reacting such compounds with anhydrides such as succinic anhydride

The compounds disclosed herein may be isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

For the purposes of further describing compounds which may be used in the present invention, following terms and definitions are provided by way of example

The term “halogen” as used herein refers to bromine, chlorine, fluorine or iodine. In one embodiment, the halogen is chlorine. In another embodiment, the halogen is bromine.

The term “hydroxy” as used herein refers to —OH.

The term “alkyl” as used herein refers to straight- or branched-chain aliphatic groups containing 1-20 carbon atoms, preferably 1-7 carbon atoms and most preferably 1-4 carbon atoms. This definition applies as well to the alkyl portion of alkoxy, alkanoyl and aralkyl groups. In one embodiment, the alkyl is a methyl group.

The term “alkoxy” includes substituted and unsubstituted alkyl, alkenyl, and alkynyl groups covalently linked to an oxygen atom. In one embodiment, the alkoxy group contains 1 to 4 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy.

The term “aryl” as used herein refers to monocyclic or bicyclic aromatic hydrocarbon groups having from 6 to 12 carbon atoms in the ring portion, for example, phenyl, naphthyl, biphenyl and diphenyl groups, each of which may be substituted with, for example, one to four substituents such as alkyl, substituted alkyl as defined above, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy. Specific embodiments of aryl groups in accordance with the present invention include phenyl, substituted phenyl, naphthyl, biphenyl, and diphenyl

The term “nitro”, as used herein alone or in combination refers to a—NO₂ group.

The term “amino” as used herein refers to the group —NRR′, where R and R′ may independently be hydrogen, alkyl, phenyl, alkoxy, or heterophenyl. The term “aminoalkyl” as used herein represents a more detailed selection as compared to “amino” and refers to the group —NRR′, where R and R′ may independently be hydrogen or (C₁-C₄)alkyl.

The term “trifluoromethyl” as used herein refers to —CF₃.

The term “trifluoromethoxy” as used herein refers to —OCF₃

The term “cycloalkyl” as used herein refers to a saturated cyclic hydrocarbon ring system containing from 3 to 7 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 7 carbon atoms in the cyclic portion and 1 to 4 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to —C(O)-alkyl groups and —O—C(O)-alkyl groups, each optionally containing 2-5 carbon atoms. Specific embodiments of alkanoyl and alkanoyloxy groups are acetyl and acetoxy, respectively.

The term “aroyl,” as used alone or in combination herein, refers to an aryl radical derived from an aromatic carboxylic acid, such as optionally substituted benzoic or naphthoic acids.

The term “aralkyl” as used herein refers to an aryl group bonded to the 4-pyridinyl ring through an alkyl group, preferably one containing 1-4 carbon atoms. A preferred aralkyl group is benzyl.

The term “nitrile” or “cyano” as used herein refers to the group —CN

The term “dialkylamino” refers to an amino group having two attached alkyl groups that can be the same or different.

The term “alkenyl” refers to a straight or branched alkenyl group of 2 to 10 carbon atoms having 1 to 3 double bonds. Preferred embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc.

The term “alkynyl” as used herein refers to a straight or branched alkynyl group of 2 to 10 carbon atoms having 1 to 3 triple bonds. Exemplary alkynyls include, but are not limited to, ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, and 2-decynyl.

The term “hydroxyalkyl” alone or in combination, refers to an alkyl group as previously defined, wherein one or several hydrogen atoms, preferably one hydrogen atom has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C₁-C₄)alkyl.

The term “alkylaminoalkyl” refers to an alkylamino group linked via an alkyl group (i.e., a group having the general structure —alkyl-NH-alkyl or —alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl, N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “carboxyalkyl” as used herein refers to the substituent —R′—COOH wherein R′ is alkylene; and carbalkoxyalkyl refers to —R′—COOR wherein R′ and R are alkylene and alkyl respectively. In certain embodiments, alkyl refers to a saturated straight- or branched-chain hydrocarbyl radical of 1-6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxyalkyl” refers to an alkylene group substituted with an alkoxy group. For example, methoxyethyl [CH₃OCH₂CH₂—] and ethoxymethyl (CH₃CH₂OCH₂—] are both C₃ alkoxyalkyl groups.

The term “carboxy”, as used herein, represents a group of the formula —COOH.

The term “alkanoylamino” refers to alkyl, alkenyl or alkynyl groups containing the group —C(O)— followed by —N(H)—, for example acetylamino, propanoylamino and butanoylamino and the like.

The term “carbonylamino” refers to the group —NR—CO—CH₂—R′, where R and R′ may be independently selected from hydrogen or (C₁-C₄)alkyl.

The term “carbamoyl” as used herein refers to —O—C(O)NH₂.

The term “carbamyl” as used herein refers to a functional group in which a nitrogen atom is directly bonded to a carbonyl, i.e., as in —NRC(═O)R′ or —C(═O)NRR′,

wherein R and R′ can be hydrogen, alkyl, substituted alkyl, alkenyl, substituted alkenyl, alkoxy, cycloalkyl, phenyl, heterocyclo, or heterophenyl.

The term “heterocyclo” refers to an optionally substituted, unsaturated, partially saturated, or fully saturated, aromatic or nonaromatic cyclic group that is a 4 to 7 membered monocyclic, or 7 to 11 membered bicyclic ring system that has at least one heteroatom in at least one carbon atom-containing ring. The substituents on the heterocyclo rings may be selected from those given above for the aryl groups. Each ring of the heterocyclo group containing a heteroatom may have 1, 2 or 3 heteroatoms selected from nitrogen atoms, oxygen atoms and sulfur atoms. Plural heteroatoms in a given heterocyclo ring may be the same or different. The heterocyclo group may be attached to the 4-pyridinyl ring at any heteroatom or carbon atom. In one embodiment, two R groups form a fused ring with the carbons at position 2 and 3 of the pyridinyl ring, there is formed a 7-quinolin-4-yl moiety.

The term “heteroaryl” refers to an optionally substituted monocyclic or bicyclic heterocyclic aryl group (i.e., an aromatic heterocyclic group) that is a 4 to 7 membered monocyclic, or 7 to 11 membered bicyclic ring system that has at least one heteroatom in at least one carbon atom-containing ring. Each ring of the heteroaryl group containing a heteroatom may have 1, 2 or 3 heteroatoms selected from nitrogen atoms, oxygen atoms and sulfur atoms. Plural heteroatoms in a given heteroaryl group may be the same or different. Specific embodiments of heteroaryl groups in accordance with the present invention include furan, thiophene, pyrrole, imidazole, pyrazole, triazole, tetrazole, thiazole, isothiazole, pyridine, pyridizine, pyrimidine, pyrazine, triazine, indole, benzofuran, benzothiophene, benzothiazole, quinoline, isoquinoline, cinnoline, phthalazine, quinazoline, quinoxaline, chromane and isochromane groups.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The methods of the instant invention are effective for treating or preventing a variety of central nervous system (CNS) disorders in a mammalian subject. Mammalian subjects amenable for treatment using these methods include, but are not limited to, human and other mammalian subjects suffering from a CNS disorder that responds positively to intervention by inhibition of biogenic amine transport. Methods are provided herein which employ an effective amount of a triple reuptake inhibitor, exemplified by the compounds described herein, to treat or prevent a selected CNS disorder in a subject. Administration of a triple reuptake inhibitor to a subject provides a therapeutic or prophylactic benefit by inhibiting or blocking native and promiscuous uptake of the biogenic amines norepinephrine, serotonin, and dopamine.

Biogenic amine reuptake inhibition in the context of the present invention can optionally be determined and selected by using one or more triple reuptake inhibitors to achieve variable selectivity and potency of transporter inhibition, wherein a combination of norepinephrine, serotonin and dopamine transporters can be inhibited, at pre-determined levels or ratios among or between different transporters. In this context, compounds useful in the present invention exhibit a wide range of potencies as inhibitors of the norepinephrine, serotonin and dopamine transporters, rendering them useful in a broad array of therapeutic applications. Accordingly, a compound useful in the invention may inhibit cellular uptake of two, or three, biogenic amine neurotransmitters non-uniformly by inhibiting uptake of norepinephrine, serotonin and/or dopamine by a factor of two- to ten, up to fifteen-fold greater than a potency of the compound for inhibiting uptake of at least one different member of the biogenic amine neurotransmitters.

The methods disclosed herein are used to treat or prevent one or more symptom(s) of a CNS disorder alleviated by inhibiting uptake of dopamine, norepinephrine, and serotonin. In certain embodiments, “treatment” or “treating” refers to amelioration of one or more symptom(s) of a CNS disorder, whereby the symptom(s) is/are alleviated by inhibiting dopamine, norepinephrine and serotonin uptake. In other embodiments, “treatment” or “treating” refers to an amelioration of at least one measurable physical parameter associated with a CNS disorder. In yet another embodiment, “treatment” or “treating” refers to inhibiting or reducing the progression or severity of a CNS disorder (or one or more symptom(s) thereof) alleviated by inhibiting dopamine, norepinephrine, and serotonin uptake, e.g., as discerned based on physical, physiological, and/or psychological parameters. In additional embodiments, “treatment” or “treating” refers to delaying the onset of a CNS disorder (or one or more symptom(s) thereof) alleviated by inhibiting dopamine, norepinephrine, and serotonin uptake.

In certain embodiments, the methods disclosed herein include administration of a triple reuptake inhibitor or a pharmaceutically acceptable salt thereof to a mammalian subject, for example a human patient, as a preventative or prophylactic treatment against a CNS disorder (or one or more symptom(s) thereof) alleviated by inhibiting dopamine, norepinephrine, and serotonin uptake. As used herein, “prevention”, “preventing”, and prophylaxis refers to a reduction in the risk or likelihood that the subject will acquire a CNS disorder or one or more symptom(s) thereof, which risk or likelihood is reduced in the subject by inhibiting dopamine, norepinephrine, and serotonin uptake. Alternatively, prevention and prophylaxis may correlate with a reduced risk of recurrence of the CNS disorder or symptom(s) thereof in the subject once the subject has been cured, restored to a normal state, or placed in remission from the subject CNS disorder. In related embodiments, a method of the invention is used as a preventative measure to the subject. Exemplary subjects amenable to prophylactic treatment in this context may have a genetic predisposition to a CNS disorder amenable to treatment by inhibiting dopamine, serotonin, and norepinephrine reuptake, such as a family history of a biochemical imbalance in the brain, or a non-genetic predisposition to a disorder alleviated by inhibiting dopamine, serotonin, and norepinephrine reuptake.

A method of the present invention is useful for treating or preventing endogenous disorders alleviated by inhibiting dopamine, norepinephrine, and serotonin uptake. Such disorders include, but are not limited to, attention-deficit disorder, depression, anxiety, obesity, Parkinson's disease, tic disorders, and addictive and substance abuse disorders.

Disorders alleviated by inhibiting dopamine, norepinephrine, and serotonin uptake are not limited to the specific disorders described herein, and the methods of the invention will be understood or readily ascertained to provide effective treatment agents for treating and/or preventing a wide range of additional CNS disorders and associated symptoms. For example, the methods of the invention will provide promising candidates for treatment and/or prevention of attention deficit hyperactivity disorder and related symptoms, as well as forms and symptoms of alcohol abuse, drug abuse, cognitive disorders, obsessive compulsive behaviors, learning disorders, reading problems, gambling addiction, manic symptoms, phobias, panic attacks, oppositional defiant behavior, conduct disorder, academic problems in school, smoking, abnormal sexual behaviors, schizoid behaviors, traumatic brain injury, somatization, depression, neuropathic pain, sleep disorders, general anxiety, stuttering, and tic disorders. Other disorders for which the compounds of the present invention may be useful include irritable bowel syndrome; inflammatory bowel disease; urinary tract disorders, such as stress urinary incontinence; PMDD (Premenstrual dysphoric disorder), degenerative diseases, including Alzheimers disease, and amyotrophic lateral sclerosis; and pyretic conditions (including fevers, and post- and peri-menopausal hot flashes). These and other symptoms, regardless of the underlying CNS disorder, are each targets for the novel methods of the invention that mediate therapeutic benefits by inhibiting dopamine, norepinephrine, and serotonin uptake. Additional CNS disorders contemplated for treatment employing the methods of the invention are described, for example, in the Quick Reference to the Diagnostic Criteria from DSM-IV (Diagnostic and Statistical Manual of Mental Disorders, Fourth Edition), The American Psychiatric Association, Washington, D.C., 1994. Cognitive disorders for treatment and/or prevention according to the invention, include, but are not limited to, Attention-Deficit/Hyperactivity Disorder, Predominately Inattentive Type; Attention-Deficit/Hyperactivity Disorder, Predominately Hyperactivity-Impulsive Type; Attention-Deficit/Hyperactivity Disorder, Combined Type; Attention-Deficit/Hyperactivity Disorder not otherwise specified (NOS); Conduct Disorder; Oppositional Defiant Disorder; Disruptive Behavior Disorder not otherwise specified (NOS); as well as cognitive deficits in depression, bipolar disorder and schizophrenia, and mild cognitive impairment.

Depressive disorders amenable for treatment and/or prevention according to the invention include, but are not limited to, Major Depressive Disorder, Recurrent; Dysthymic Disorder; Depressive Disorder not otherwise specified (NOS); and Major Depressive Disorder, Single Episode.

CNS disorders involving chronic pain states are amenable for treatment and/or prevention according to the invention. Such chronic pain states include, but are not limited to, neuropathic pain, fibromyalgia, traumatic brain injury, substance abuse, and irritable bowel syndrome. Neuropathic pain can arise from or be associated with a wide range of conditions, including, but not limited to, viral neuralgias (e.g., herpes, AIDS), diabetic neuropathy, phantom limb pain, stump/neuroma pain, post-ischemic pain (stroke), fibromyalgia, reflex sympathetic dystrophy (RSD), complex regional pain syndrome (CRPS), cancer pain, back pain, vertebral disk rupture, and trigeminal neuralgia, and cancer-chemotherapy-induced neuropathic pain.

Addictive disorders amenable for treatment and/or prevention employing the methods and compositions of the invention include, but are not limited to, eating disorders, impulse control disorders, alcohol-related disorders, nicotine-related disorders, amphetamine-related disorders, cannabis-related disorders, cocaine-related disorders, hallucinogen use disorders, inhalant-related disorders, and opioid-related disorders, all of which are further sub-classified as listed below.

Eating disorders include, but are not limited to, Bulimia Nervosa, Nonpurging Type; Bulimia Nervosa, Purging Type; and Eating Disorder not otherwise specified (NOS).

Impulse control disorders include, but are not limited to, Intermittent Explosive Disorder, Kleptomania, Pyromania, Pathological Gambling, Trichotillomania, and Impulse Control Disorder not otherwise specified (NOS).

Alcohol-related disorders include, but are not limited to, Alcohol-Induced Psychotic Disorder, with delusions; Alcohol Abuse; Alcohol Intoxication; Alcohol Withdrawal; Alcohol Intoxication Delirium; Alcohol Withdrawal Delirium; Alcohol-Induced Persisting Dementia; Alcohol-Induced Persisting Amnestic Disorder; Alcohol Dependence; Alcohol-Induced Psychotic Disorder, with hallucinations; Alcohol-Induced Mood Disorder; Alcohol-Induced Anxiety Disorder; Alcohol-Induced Sexual Dysfunction; Alcohol-Induced Sleep Disorders; Alcohol-Related Disorders not otherwise specified (NOS); Alcohol Intoxication; and Alcohol Withdrawal.

Nicotine-related disorders include, but are not limited to, Nicotine Dependence, Nicotine Withdrawal, and Nicotine-Related Disorder not otherwise specified (NOS).

Amphetamine-related disorders include, but are not limited to, Amphetamine Dependence, Amphetamine Abuse, Amphetamine Intoxication, Amphetamine Withdrawal, Amphetamine Intoxication Delirium, Amphetamine-Induced Psychotic Disorder with delusions, Amphetamine-Induced Psychotic Disorders with hallucinations, Amphetamine-Induced Mood Disorder, Amphetamine-Induced Anxiety Disorder, Amphetamine-Induced Sexual Dysfunction, Amphetamine-Induced Sleep Disorder, Amphetamine Related Disorder not otherwise specified (NOS), Amphetamine Intoxication, and Amphetamine Withdrawal.

Cannabis-related disorders include, but are not limited to, Cannabis Dependence; Cannabis Abuse; Cannabis Intoxication; Cannabis Intoxication Delirium; Cannabis-Induced Psychotic Disorder, with delusions; Cannabis-Induced Psychotic Disorder with hallucinations; Cannabis-Induced Anxiety Disorder; Cannabis Related Disorder not otherwise specified (NOS); and Cannabis Intoxication.

Cocaine-related disorders include, but are not limited to, Cocaine Dependence, Cocaine Abuse, Cocaine Intoxication, Cocaine Withdrawal, Cocaine Intoxication Delirium, Cocaine-Induced Psychotic Disorder with delusions, Cocaine-Induced Psychotic Disorders with hallucinations, Cocaine-Induced Mood Disorder, Cocaine-Induced Anxiety Disorder, Cocaine-Induced Sexual Dysfunction, Cocaine-Induced Sleep Disorder, Cocaine Related Disorder not otherwise specified (NOS), Cocaine Intoxication, and Cocaine Withdrawal.

Hallucinogen-use disorders include, but are not limited to, Hallucinogen Dependence, Hallucinogen Abuse, Hallucinogen Intoxication, Hallucinogen Withdrawal, Hallucinogen Intoxication Delirium, Hallucinogen-Induced Psychotic Disorder with delusions, Hallucinogen-Induced Psychotic Disorders with hallucinations, Hallucinogen-Induced Mood Disorder, Hallucinogen-Induced Anxiety Disorder, Hallucinogen-Induced Sexual Dysfunction, Hallucinogen-Induced Sleep Disorder, Hallucinogen Related Disorder not otherwise specified (NOS), Hallucinogen Intoxication, and Hallucinogen Persisting Perception Disorder (Flashbacks).

Inhalant-related disorders include, but are not limited to, Inhalant Dependence; Inhalant Abuse; Inhalant Intoxication; Inhalant Intoxication Delirium; Inhalant-Induced Psychotic Disorder, with delusions; Inhalant-Induced Psychotic Disorder with hallucinations; Inhalant-Induced Anxiety Disorder; Inhalant Related Disorder not otherwise specified (NOS); and Inhalant Intoxication.

Opioid-related disorders include, but are not limited to, Opioid Dependence, Opioid Abuse, Opioid Intoxication, Opioid Intoxication Delirium, Opioid-Induced Psychotic Disorder with delusions, Opioid-Induced Psychotic Disorder with hallucinations, Opioid-Induced Anxiety Disorder, Opioid Related Disorder not otherwise specified (NOS), Opioid Intoxication, and Opioid Withdrawal.

Tic disorders include, but are not limited to, Tourette's Disorder, Chronic Motor or Vocal Tic Disorder, Transient Tic Disorder, Tic Disorder not otherwise specified (NOS), Stuttering, Autistic Disorder, and Somatization Disorder.

By virtue of inhibiting native and promiscuous uptake, the novel methods of the present invention are thus useful in a wide range of veterinary and human medical applications, in particular for treating and/or preventing a wide array of CNS disorders and/or associated symptom(s) alleviated by inhibiting dopamine, norepinephrine and serotonin uptake.

Furthermore, the methods of the present invention are effective in the treatment of those who have been previously treated for disorders affected by monoamine neurotransmitters such as depression. The methods described herein are additionally effective in the treatment of those who have had refractory experiences with prior treatments, i.e. individuals who have not responded, responded insufficiently, been unable to tolerate previous treatment(s) or who have otherwise responded in an unsatisfactory manner to other medications affecting monoamine neurotransmitters such as anti-depressants including, but not limited to, tri-cyclic antidepressants (TCAs), specific monoamine reuptake inhibitors, selective serotonin reuptake inhibitors, selective norepinephrine or noradrenaline reuptake inhibitors, selective dopamine reuptake inhibitors, serotonin-norepinephrine reuptake inhibitors, norepinephrine-dopamine reuptake inhibitors, monoamine oxidase inhibitors, atypical antidepressants, atypical antipsychotics, anticonvulsants, or opiate agonists. Individuals may have been refractory to previous treatment(s) for any reason. In some embodiments, refractory individuals may have failed to respond or failed to respond sufficiently to a previous treatment. In one embodiment, a refractory individual may have treatment resistant depression. In other embodiments, a refractory individual may have responded to the initial treatment, but not succeed in entering remission from the treatment. In some embodiments, refractory individuals may have been unable to continue taking the medication due to intolerance of the medication including side effects such as, but not limited to, sexual dysfunction, weight gain, insomnia, dry mouth, constipation, nausea and vomiting, dizziness, memory loss, agitation, anxiety, sedation, headache, urinary retention, or abdominal pain.

Within additional aspects of the invention, methods are provided that employ combinatorial formulations and coordinate administration of an effective amount of a triple reuptake inhibitor compound (or a pharmaceutically effective enantiomer, salt, solvate, hydrate, polymorph, or prodrug thereof), and one or more additional active agent(s) that is/are combinatorially formulated or coordinately administered with a triple reuptake inhibitor, such as the compounds described herein—yielding a combinatorial formulation or coordinate administration method that is effective to modulate, alleviate, treat or prevent a targeted CNS disorder, or one or more symptom(s) thereof, in a mammalian subject. Exemplary combinatorial formulations and coordinate treatment methods in this context comprise a therapeutic compound as described herein in combination with one or more additional or adjunctive treatment agents or methods for treating the targeted CNS disorder or symptom(s), for example one or more antidepressant or anxiolytic agent(s) and/or therapeutic method(s).

In related embodiments of the methods of the invention, a triple reuptake inhibitor compound can be used in combination therapy with at least one other therapeutic agent or method. In this context, a triple reuptake inhibitor, such as the compounds disclosed herein, can be administered concurrently or sequentially with administration of a second therapeutic agent. For example, a second agent that acts to treat or prevent the same, or different, CNS disorder or symptom(s) for which the triple reuptake inhibitor compound is administered. The triple reuptake inhibitor compound and the second therapeutic agent can be combined in a single composition or administered in different compositions. The second therapeutic agent may also be effective for treating and/or preventing a CNS disorder or associated symptom(s) by inhibiting dopamine and/or norepinephrine and/or serotonin uptake. The coordinate administration may be done simultaneously or sequentially in either order, and there may be a time period while only one or both (or all) active therapeutic agents, individually and/or collectively, exert their biological activities and therapeutic effects. A distinguishing aspect of all such coordinate treatment methods is that the triple reuptake inhibitor compound exerts at least some detectable therapeutic activity toward alleviating or preventing the targeted CNS disorder or symptom(s), as described herein, and/or elicit a favorable clinical response, which may or may not be in conjunction with a secondary clinical response provided by the secondary therapeutic agent. Often, the coordinate administration of a triple reuptake inhibitor compound with a secondary therapeutic agent as contemplated herein will yield an enhanced therapeutic response beyond the therapeutic response elicited by either or both the triple reuptake inhibitor compound and/or secondary therapeutic agent alone.

As many of the CNS disorders and symptoms treatable or preventable using compounds of the present invention are chronic, in one embodiment combination therapy involves alternating between administering a triple reuptake inhibitor compound and a second therapeutic agent (i.e., alternating therapy regimens between the two drugs, e.g., at one week, one month, three month, six month, or one year intervals). Alternating drug regimens in this context will often reduce or even eliminate adverse side effects, such as toxicity, that may attend long-term administration of one or both drugs alone.

In certain embodiments of the invention, the additional or secondary psychotherapeutic agent is an anti-depressant drug, which may include, for example, any species within the broad families of tricyclic anti-depressants (TCAs) including, but not limited to, doxepin, clomipramine, amitriptyline, maprotiline, imipramine, nortryptyline, trimipramine, protriptyline, amoxapine, and desipramine; specific monoamine reuptake inhibitors; selective serotonin reuptake inhibitors (SSRIs) including, but not limited to, citalopram, escitalopram, fluoxetine, fluvoxamine, sertraline, vilazodone, and paroxetine; selective norepinephrine reuptake inhibitors; selective dopamine reuptake inhibitors; monoamine oxidase inhibitors (MAOIs) such as phenelzine, nortriptyline, selegiline, nefazodone, and tranylcypromine; norepinephrine reuptake inhibitors (NRIs); tetracyclic anti-depressants such as mirtazapine; vilazodone, agomelatine, multiple monoamine reuptake inhibitors, e.g., that inhibit both serotonin and norepinephrine reuptake (SNRIs) including, but not limited to, venlafaxine, desvenlafaxine, and duloxetine, and indeterminate (atypical) anti-depressants.

The additional or secondary psychotherapeutic agent may additionally include atypical antipsychotics including, but not limited to, aripiprazole, ziprasidone, risperidone, quetiepine, or olanzapine or anticonvulsants including but not limited to lamotrigine, carbamazepine, oxcarbazepine, valproate, levetriacetam, and topiramate. Psychotherapeutic agents may additionally include opiate agonists including, but not limited to, buprenorphine, methadone and LAAM. The additional or secondary psychotherapeutic agent may further include atomoxetine; dothiepin; isocarboxazid; lofepramine; maprotiline; milnacipran; moclobemide; quetiapine; reboxetine; tianeptine; and trazodone.

In other detailed combinatorial formulations and coordinate treatment methods of the present invention, the additional or secondary psychotherapeutic agent is an anxiolytic drug agent including, but not limited to, benzodiazepines, such as alaprazolam, chlordiazepoxide, clonazepam, chlorazepate, diazepam, lorazepam, oxazepam and prazepam; non-benzodiazepine agents, such as buspirone; and tranquilizers, such as barbituates.

In other embodiments of combinatorial formulations and coordinate treatment methods provided herein, the secondary therapeutic agent is an anti-attention-deficit-disorder treatment agent. Examples of useful anti-attention-deficit-disorder agents for use in these embodiments include, but are not limited to, methylphenidate; dextroamphetamine and dextroamphetamine salts and prodrugs; tricyclic antidepressants, such as imipramine, desipramine, and nortriptyline; and psychostimulants, such as pemoline and deanol.

In additional embodiments of combinatorial formulations and coordinate treatment methods provided herein, the secondary therapeutic agent is an anti-addictive-disorder agent. Examples of useful anti-addictive-disorder agents include, but are not limited to, tricyclic antidepressants; glutamate antagonists, such as ketamine HCl, dextromethorphan, dextrorphan tartrate and dizocilpine (MK801); degrading enzymes, such as anesthetics and aspartate antagonists; GABA agonists, such as baclofen and muscimol HBr; reuptake blockers; degrading enzyme blockers; glutamate agonists, such as D-cycloserine, carboxyphenylglycine, L-glutamic acid, and cis-piperidine-2,3-dicarboxylic acid; aspartate agonists; GABA antagonists such as gabazine (SR-95531), saclofen, bicuculline, picrotoxin, and (+) apomorphine HCl; and dopamine antagonists, such as spiperone HCl, haloperidol, and (−) sulpiride.

In other embodiments of combinatorial formulations and coordinate treatment methods provided herein, the secondary therapeutic agent is an anti-alcohol agent. Examples of useful anti-alcohol agents include, but are not limited to, disulfuram and naltrexone.

In other embodiments of combinatorial formulations and coordinate treatment methods provided herein, the secondary therapeutic agent is an anti-nicotine agent. Examples of useful anti-nicotine agents include, but are not limited to, clonidine.

In other embodiments of combinatorial formulations and coordinate treatment methods provided herein, the secondary therapeutic agent is an anti-opiate agent. Examples of useful anti-opiate agents include, but are not limited to, methadone, clonidine, lofexidine, levomethadyl acetate HCl, naltrexone, and buprenorphine.

In other embodiments of combinatorial formulations and coordinate treatment methods provided herein, the secondary therapeutic agent is anti-cocaine agent. Examples of useful anti-cocaine agents include, but are not limited to, desipramine, amantadine, fluoxidine, and buprenorphine.

In other embodiments of combinatorial formulations and coordinate treatment methods provided herein, the secondary therapeutic agent is an anti-lysergic acid diethylamide (“anti-LSD”) agent. Examples of useful anti-LSD agents include, but are not limited to, diazepam.

In other embodiments of combinatorial formulations and coordinate treatment methods provided herein, the secondary therapeutic agent is an anti-phencyclidine (“anti-PCP”) agent. Examples of useful anti-PCP agents include, but are not limited to, haloperidol.

In other embodiments of combinatorial formulations and coordinate treatment methods provided herein, the secondary therapeutic agent is an appetite suppressant. Examples of useful appetite suppressants include, but are not limited to, fenfluramine, phenylpropanolamine, and mazindol.

In yet additional embodiments of combinatorial formulations and coordinate treatment methods provided herein, the secondary therapeutic agent is an anti-Parkinson's-disease agent. Examples of useful anti-Parkinson's-disease agents include, but are not limited to, ropinirole; pramipexole; dopamine precursors, such as levodopa (L-DOPA), L-phenylalanine, and L-tyrosine; neuroprotective agents; dopamine agonists; dopamine reuptake inhibitors; anticholinergics such as amantadine and memantine; and 1,3,5-trisubstituted adamantanes, such as 1-amino-3,5-dimethyl-adamantane. (See, U.S. Pat. No. 4,122,193).

In particular, use of a triple reuptake inhibitor could advantageously be used in combination with levodopa for treatment of Parkinson's disease. Without being bound to a specific mechanism of action, the blocking of promiscuous uptake would likely decrease the uptake or clearance of L-DOPA-derived dopamine into serotonin, dopamine, and norepinephrine neurons. Accordingly, the effectiveness of L-DOPA would be enhanced, allowing for a reduction in the dosage of L-DOPA required and potentially reducing adverse L-DOPA events such as dyskinesias. Moreover, blocking of promiscuous uptake would enhance the effect of norepinephrine, providing for treatment of common Parkinson's disease co-morbidities such as depression and cognitive dysfunction.

In further combinatorial formulations and coordinate treatment methods of the present invention, the additional or secondary psychotherapeutic agent is a stimulant including, but not limited to, modafinil, methylphenidate, dextroamphetamine, and methamphetamine. Sodium oxybate may also be used in the treatment of narcolepsy.

In additional combinatorial formulations and coordinate treatment methods of the present invention, the additional or secondary psychotherapeutic agent is a muscle relaxant or sleep medication, including, but not limited to, clonazepam, triazolam, eszopiclone, ramelteon, temazepam, zaleplon and zolpidem.

In yet another embodiment of the invention, combinatorial formulations and coordinate treatment methods of the present invention the additional or secondary psychotherapeutic agent comprises an anti-epileptics including, but not limited to, gabapentin.

In further combinatorial formulations and coordinate treatment methods of the present invention, the additional psychotherapeutic agent is an opioid, including but not limited to, codeine, oxycodone and hydrocodone.

In yet another combinatorial formulation and coordinate treatment method of the present invention, the additional or secondary psychotherapeutic agent is clonidine or guanfacine.

Additionally contemplated for use herein are compounds that have multiple modes of action, for example, selective serotonin reuptake inhibitors and 5HT1a agonists and partial agonists such as, but not limited to vilazodone.

Administration of an effective amount of triple reuptake inhibitor in the methods described herein to a mammalian subject presenting with one or more symptoms of a CNS disorder or other neurological or psychiatric condition will detectably decrease, eliminate, or prevent the targeted CNS disorder and/or associated symptom(s). In exemplary embodiments, administration of a triple reuptake inhibitor composition to a suitable test subject will yield a reduction in one or more target symptom(s) associated with a selected CNS disorder, such as pain, by at least 10%, 20%, 30%, 50% or greater, up to a 75-90%, or 95% or greater, reduction in the targeted CNS disorder or one or more target symptom(s), compared to placebo-treated or other suitable control subjects. Comparable levels of efficacy are contemplated for the entire range of CNS disorders, including all contemplated neurological and psychiatric disorders, and related conditions and symptoms, for treatment or prevention using the methods of the invention

An “effective amount,” “therapeutic amount,” “therapeutically effective amount,” or “effective dose” of triple reuptake inhibitor agent and/or a psychotherapeutic agent as used herein means an effective amount or dose of the active compound as described herein sufficient to elicit a desired pharmacological or therapeutic effect in a human subject. Such an effect typically results in a measurable reduction in an occurrence, frequency, or severity of one or more symptom(s) of a CNS disorder, including any combination of neurological and/or psychological symptoms, diseases, or conditions, associated with or caused by the targeted CNS disorder, in the subject. In certain embodiments, when a compound as described herein is administered to treat a CNS disorder, for example a pain disorder, an effective amount of the compound will be an amount sufficient in vivo to delay or eliminate onset of symptoms of the targeted condition or disorder.

Therapeutic efficacy can alternatively be demonstrated by a decrease in the frequency or severity of symptoms associated with the treated central nervous system condition or disorder, or by altering the nature, occurrence, recurrence, or duration of symptoms associated with the treated condition or disorder. In this context, “effective amounts,” “therapeutic amounts,” “therapeutically effective amounts,” and “effective doses” of triple reuptake inhibitor agents described herein can be readily determined by ordinarily skilled artisans following the teachings of this disclosure and employing tools and methods generally known in the art, often based on routine clinical or patient-specific factors.

In the case of antidepressant therapeutic agents, these terms most often refer to a measurable, statistically significant reduction in an occurrence, frequency, or severity of one or more symptom(s) of a specified central nervous system disorder, including any combination of neurological and/or psychological symptoms, diseases, or conditions, associated with or caused by the targeted central nervous system disorder and/or reduction in the development of depression in a target population.

Efficacy of the treatment methods of the invention will often be determined by use of conventional patient surveys or clinical scales to measure clinical indices of disorders in subjects. The methods of the invention will yield a reduction in one or more scores or selected values generated from such surveys or scales completed by test subjects (indicating for example an incidence or severity of a selected anxiety disorder), by at least 10%, 20%, 30%, 50% or greater, up to a 75-90%, or 95% compared to correlative scores or values observed for control subjects treated with placebo or other suitable control treatment. In at risk populations, the methods of the invention will yield a stable or minimally variable change in one or more scores or selected values generated from such surveys or scales completed by test subjects. More detailed data regarding efficacy of the methods and compositions of the invention can be determined using alternative clinical trial designs.

Useful patient surveys and clinical scales for comparative measurement of clinical indices of psychiatric disorders in subjects treated using the methods and compositions of the invention can include any of a variety of widely used and well known surveys and clinical scales. Among these useful tools are the Mini International Neuropsychiatric Interview© (MINI) (Sheehan et al., 1998); Clinical Global Impression scale (CGI) (Guy, W., ECDEU Assessment Manual for Psychopharmacology, DHEW Publication No. (ADM) 76-338, rev. 1976); HAM-A rating scale for anxiety (Hamilton, 1959); Clinician-Administered Post-traumatic Stress Disorder Scale (CAPS) (Weathers et al., 1999); Clinician-Administered PTSD Scale Part 2 (CAPS-2) (Blake et al., 1995); Clinician-Administered PTSD Scale for Children and Adolescents (CAPS-CA) (Nader et al., 1996); Impact of Event Scale (IES) (Horowitz et al. 1979); Impact of Event Scale-Revised (IES-R) (Weiss et al. 1996); Clinical Global Impression Severity of Illness (CGI-S) (Guy, 1976); Clinical Global Impression Improvement (CGI-I) (Guy, et al. 1976); Duke Global Rating for PTSD scale (DGRP) (Davidson et al., 1998); Duke Global Rating for PTSD scale Improvement (DGRP-I); Structured Interview for PTSD (SI-PTSD) (Davidson, et al. 1990); PTSD Interview (PTSD-I) (Watson et al., 1991); PTSD Symptom Scale (PSS-I) (Foa et al., 2006); Beck Depression Inventory (BDI) (Beck, 2006); Revised Hamilton Rating Scale for Depression (RHRSD) (Warren, 1994); Major Depressive Inventory (MDI) (Olsen et al. 2003); and Children's Depression Index (CDI) (Kovacs, et al. 1981).

Any of these scales, alone or in combination, can be effectively employed to determine efficacy of the methods of the invention. Additionally, a variety of other scales and methods for assessing comparative anxiety disorder symptoms or status, are widely used and well known in the art for use within the invention. Other exemplary scales for assessing efficacy of the invention include, for example, the Hamilton Depression Rating Scale© (HDRS) (Hamilton, M., J. Neurol. Neurosurg. Psychiatr. 23:56-62, 1960; Hamilton, M., Br. J. Soc. Clin. Psychol. 6:278-296, 1967); Montgomery-Asberg Depression Rating Scale© (MADRS) (Montgomery and Asberg, 1979); Beck Scale for Suicide Ideation® (BSS) (Beck and Steer, 1991 Columbia-Suicide Severity Rating Scale© (C-SSRS) or Columbia Classification Algorithm of Suicide Assessment© (C CASA) (Posner, K, et al., 2007); Sheehan-Suicidality Tracking Scale© (S-SST) (Conic et al., 2009); Beck Hopelessness Scale© (BHS) (Beck, Steer, 1988); Geriatric Depression Scale (GDS) (Yesavage, J. A. et al., J. Psychiatr. Res. 17:37-49, 1983). HAM-D scale for depression (Hamilton, 1960); the Yale-Brown Obsessive Compulsive Scale (YBOCS) (Goodman et al., 1989); The Positive and Negative Syndrome Scale (PANSS) for schizophrenia (Kay et al., 1987); the YMRS rating scale for mania (Young et al., 1978); the Liebowitz Social Anxiety Scale (Heimburg et al., 2002).

The methods of the invention will yield a reduction in one or more scores or values generated from these clinical surveys (using any single scale or survey, or any combination of one or more of the surveys described above) by at least 10%, 20%, 30%, 50% or greater, up to a 75-90%, or 95% compared to correlative scores or values observed for control subjects treated with placebo or other suitable control treatment. In prophylactic treatment, the methods of the invention will yield a stabilization or diminished change in the scores or values generated from these clinical surveys. For example, the Clinical Global Impression (CGI) scale is a 7-point, clinician rated scale to determine severity, improvement and response to treatment for selected anxiety disorders. The CGI severity of illness scale uses a range of responses from 1 to 7, with 1 being “normal” and 7 “amongst the most severely ill patients” (Guy, 1976). A “responder” according to this measuring tool is defined as being “Much Improved” or “Very Much Improved”, having a CGI score of at least 2. Thus in one alternate expression of efficacy of the invention, a frequency of normal to moderately symptomatic CGI scores, for example scores of 1, 2, 3, or 4, will occur more often in subjects treated according to the invention, by a factor of at least 10%, 20%, 30%, 50% or greater, up to a 75-90%, or 95% compared to a frequency of the same normal to moderately symptomatic scores or values observed for control subjects completing the CGI following administration of placebo.

Suitable routes of administration for a triple reuptake inhibitor agent in the methods disclosed herein include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a triple reuptake inhibitor compound used as disclosed herein for mammalian subjects may range from about 1 to about 1800 mg, about 10 to about 1800 mg, 25 to about 1800 mg, about 50 to about 1000 mg, about 75 to about 900 mg, about 100 to about 750 mg, or about 150 to about 500 mg. In certain embodiments, the effective dosage will be selected within narrower ranges of, for example, about 5 to about 10 mg, 10 to about 25 mg, about 30 to about 50 mg, about 10 to about 300 mg, about 25 to about 300 mg, about 50 to about 100 mg, about 100 to about 250 mg, or about 250 to about 500 mg. These and other effective unit dosage amounts may be administered in a single dose, or in the form of multiple daily, weekly or monthly doses, for example in a dosing regimen comprising from 1 to 4, or 2-3, doses administered per day, per week, or per month. In exemplary embodiments, dosages of about 10 to about 25 mg, about 30 to about 50 mg, about 25 to about 150, about 50 to about 100 mg, about 100 to about 250 mg, or about 250 to about 500 mg, are administered one, two, three, or four times per day. In more detailed embodiments, dosages of about 50-75 mg, about 100-200 mg, about 250-400 mg, or about 400-600 mg are administered once or twice daily. In further detailed embodiments, dosages of about 50-100 mg are administered twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day

The amount, timing, and mode of delivery of compositions comprising an effective amount of triple reuptake inhibitor agent as used in the methods described herein will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neurological or psychiatric condition in the subject, as described herein. Thus, following administration of a triple reuptake inhibitor compound or pharmaceutically acceptable salt thereof according to the formulations and methods herein, test subjects will exhibit a 10%, 20%, 30%, 50% or greater reduction, up to a 75-90%, or 95% or greater, reduction, in one or more symptoms associated with a targeted monoamine neurotransmitter influenced disorder or other neurological or psychiatric condition, compared to placebo-treated or other suitable control subjects.

Pharmaceutical dosage forms of a compound used in the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

Pharmaceutical dosage forms of a triple reuptake inhibitor composition may include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like.

Within various combinatorial or coordinate treatment methods disclosed herein, the additional psychotherapeutic agent and triple reuptake inhibitor compound or a pharmaceutically acceptable salt thereof may each be administered by any of a variety of delivery routes and modes, which may be the same or different for each agent.

An additional psychotherapeutic compound and/or a and triple reuptake inhibitor administered according to the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate.

A triple reuptake inhibitor as disclosed herein can be prepared and administered in any of a variety of inhalation or nasal delivery forms known in the art. Devices capable of depositing aerosolized formulations of a triple reuptake inhibitor compound or a pharmaceutically acceptable salt thereof of the invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of depression. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound of the present invention, and any additional active or inactive ingredient(s).

Intranasal delivery permits the passage of active compounds as disclosed herein into the blood stream directly after administering an effective amount of the compound to the nose, without requiring the product to be deposited in the lung. In addition, intranasal delivery can achieve direct, or enhanced, delivery of the active compound to the central nervous system. In these and other embodiments, intranasal administration of the compounds of the invention may be advantageous for treating disorders influenced by monoamine neurotransmitters, by providing for rapid absorption and delivery.

For intranasal and pulmonary administration, a liquid aerosol formulation will often contain an active compound as described herein combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional methods of the invention are provided for topical administration of a triple reuptake inhibitor compound or a pharmaceutically acceptable salt thereof. Topical compositions may comprise a compound as described herein and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound as disclosed herein dissolved or dispersed in water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours.

Yet additional formulations of a compound used in the present invention are provided for parenteral administration, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; dispersions; and emulsions. The formulations may be presented in unit-dose or multi-dose containers. Pharmaceutically acceptable formulations and ingredients will typically be sterile or readily sterilizable, biologically inert, and easily administered. Parenteral preparations typically contain buffering agents and preservatives, and may be lyophilized for reconstitution at the time of administration.

Parental formulations may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

Within exemplary compositions and dosage forms used in the methods of the invention, a triple reuptake inhibitor compound or a pharmaceutically acceptable salt thereof for treating disorders disclosed herein is administered in an extended release or sustained release formulation. In these formulations, the sustained release composition of the formulation provides therapeutically effective plasma levels of the active compound (1R,5S)— or a pharmaceutically acceptable salt thereof over a sustained delivery period of approximately 8 hours or longer, or over a sustained delivery period of approximately 18 hours or longer, up to a sustained delivery period of approximately 24 hours or longer.

In exemplary embodiments, a triple reuptake inhibitor compound or a pharmaceutically acceptable salt thereof is combined with a sustained release vehicle, matrix, binder, or coating material. As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings.

In one aspect, the current invention comprises methods using an oral sustained release dosage composition for administering a triple reuptake inhibitor compound or a pharmaceutically acceptable salt thereof. In a related aspect, the invention comprises a method of reducing one or more side effects that attend administration of an oral dosage form of a triple reuptake inhibitor compound or a pharmaceutically acceptable salt thereof by employing a sustained release formulation. Within this method, following oral administration of a triple reuptake inhibitor compound or a pharmaceutically acceptable salt thereof, the active agent is released in a sustained, delayed, gradual or modified release delivery mode into the gastrointestinal tract (e.g., the intestinal lumen) of the subject over a period of hours, during which the triple reuptake inhibitor compound or a pharmaceutically acceptable salt thereof reaches, and is sustained at, a therapeutic concentration in a blood plasma, tissue, organ or other target site of activity (e.g., a central nervous system tissue, fluid or compartment) in the patient. When following this method, the side effect profile of triple reuptake inhibitor compound or a pharmaceutically acceptable salt thereof is less than a side effect profile of an equivalent dose of a triple reuptake inhibitor compound or a pharmaceutically acceptable salt thereof administered in an immediate release oral dosage form.

In certain embodiments, a triple reuptake inhibitor compound or a pharmaceutically acceptable salt thereof is released from the sustained release compositions and dosage forms of the invention and delivered into the blood plasma or other target site of activity in the subject at a sustained therapeutic level over a period of at least about 6 hours, often over a period of at least about 8 hours, at least about 12 hours, or at least about 18 hours, and in other embodiments over a period of about 24 hours or greater. By sustained therapeutic level is meant a plasma concentration level of at least a lower end of a therapeutic dosage range as exemplified herein. In more detailed embodiments of the invention, the sustained release compositions and dosage forms will yield a therapeutic level of a triple reuptake inhibitor compound or a pharmaceutically acceptable salt thereof following administration to a mammalian subject in a desired dosage amount (e.g., 5, 10, 25, 50, 100 200, 400, 600, or 800 mg) that yields a minimum plasma concentration of at least a lower end of a therapeutic dosage range as exemplified herein over a period of at least about 6 hours, at least about 8 hours, at least about 12 hours, at least about 18 hours, or up to 24 hours or longer. In alternate embodiments of the invention, the sustained release compositions and dosage forms will yield a therapeutic level of a triple reuptake inhibitor compound or a pharmaceutically acceptable salt thereof following administration to a mammalian subject in a desired dosage amount (e.g., 5, 10, 25, 50, 100, 200, 400, 600, or 800 mg) that yields a minimum plasma concentration that is known to be associated with clinical efficacy, over a period of at least about 6 hours, at least about 8 hours, at least about 12 hours, at least about 18 hours, or up to 24 hours or longer.

In certain embodiments, a triple reuptake inhibitor compound or a pharmaceutically acceptable salt thereof is released from the compositions and dosage forms disclosed herein and delivered into the blood plasma or other target site of activity in the subject (including, but not limited to, areas of the brain such as the prefrontal cortex, frontal cortex, thalamus, striatum, ventral tegmental area, other cortical areas, hippocampus, hypothalamus, or nucleus accumbens) in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In more detailed embodiments of the invention, compositions and oral dosage forms of a triple reuptake inhibitor compound or a pharmaceutically acceptable salt thereof are provided, wherein the compositions and dosage forms, after ingestion, provide a curve of concentration of a triple reuptake inhibitor compound or a pharmaceutically acceptable salt thereof agents over time, the curve having an area under the curve (AUC) which is approximately proportional to the dose of the triple reuptake inhibitor compound or a pharmaceutically acceptable salt thereof administered, and a maximum concentration (Cₘₐₓ) that is proportional to the dose of the triple reuptake inhibitor compound or a pharmaceutically acceptable salt thereof administered.

In other detailed embodiments, the Cₘₐₓ of a triple reuptake inhibitor compound or a pharmaceutically acceptable salt thereof provided after oral delivery of a composition or dosage form of the invention is less than about 80%, often less than about 75%, in some embodiments less than about 60%, or 50%, of a Cₘₐₓ obtained after administering an equivalent dose of the active compound in an immediate release oral dosage form.

Within exemplary embodiments of the invention, the compositions and dosage forms containing of a triple reuptake inhibitor compound or a pharmaceutically acceptable salt thereof and a sustained release vehicle, matrix, binder, or coating will yield sustained delivery of the active compound such that, following administration of the composition or dosage form to a mammalian treatment subject, the Cₘₐₓ of the triple reuptake inhibitor compound or a pharmaceutically acceptable salt thereof in the treatment subject is less than about 80% of a Cₘₐₓ provided in a control subject after administration of the same amount of the triple reuptake inhibitor compound or a pharmaceutically acceptable salt thereof in an immediate release formulation.

As used herein, the term “immediate release dosage form” refers to a dosage form of a triple reuptake inhibitor compound or a pharmaceutically acceptable salt thereof wherein the active compound readily dissolves upon contact with a liquid physiological medium, for example phosphate buffered saline (PBS) or natural or artificial gastric fluid. In certain embodiments, an immediate release formulation will be characterized in that at least 70% of the active compound will be dissolved within a half hour after the dosage form is contacted with a liquid physiological medium. In alternate embodiments, at least 80%, 85%, 90% or more, or up to 100%, of the active compound in an immediate release dosage form will dissolve within a half hour following contact of the dosage form with a liquid physiological medium in an art-accepted in vitro dissolution assay. These general characteristics of an immediate release dosage form will often relate to powdered or granulated compositions of a triple reuptake inhibitor compound or a pharmaceutically acceptable salt thereof in a capsulated dosage form, for example in a gelatin-encapsulated dosage form, where dissolution will often be relatively immediate after dissolution/failure of the gelatin capsule. In alternate embodiments, the immediate release dosage form may be provided in the form of a compressed tablet, granular preparation, powder, or even liquid dosage form, in which cases the dissolution profile will often be even more immediate (e.g., wherein at least 85%-95% of the active compound is dissolved within a half hour).

In additional embodiments of the invention, an immediate release dosage form will include compositions wherein the triple reuptake inhibitor compound or a pharmaceutically acceptable salt thereof is not admixed, bound, coated or otherwise associated with a formulation component that substantially impedes in vitro or in vivo dissolution and/or in vivo bioavailability of the active compound. Within certain embodiments, a triple reuptake inhibitor compound or a pharmaceutically acceptable salt thereof will be provided in an immediate release dosage form that does not contain significant amounts of a sustained release vehicle, matrix, binder or coating material. In this context, the term “significant amounts of a sustained release vehicle, matrix, binder or coating material” is not intended to exclude any amount of such materials, but an amount sufficient to impede in vitro or in vivo dissolution of a triple reuptake inhibitor compound or a pharmaceutically acceptable salt thereof in a formulation containing such materials by at least 5%, often at least 10%, and up to at least 15%-20% compared to dissolution of the triple reuptake inhibitor compound or a pharmaceutically acceptable salt thereof when provided in a composition that is essentially free of such materials.

In alternate embodiments of the invention, an immediate release dosage form of a triple reuptake inhibitor compound or a pharmaceutically acceptable salt thereof may be any dosage form comprising the active compound which fits the FDA Biopharmaceutics Classification System (BCS) Guidance definition (see, e.g., http://www.fda.gov/cder/OPS/BCS_guidance.htm) of a “high solubility substance in a rapidly dissolving formulation.” In exemplary embodiments, an immediate release formulation of a triple reuptake inhibitor compound or a pharmaceutically acceptable salt thereof according to this aspect of the invention will exhibit rapid dissolution characteristics according to BCS Guidance parameters, such that at least approximately 85% of triple reuptake inhibitor compound or a pharmaceutically acceptable salt thereof in the formulation will go into a test solution within about 30 minutes at pH 1, pH 4.5, and pH 6.8.

The compositions, dosage forms and methods disclosed herein thus include novel tools for coordinate treatment of disorders involving monoamine neurotransmitters by providing for sustained release and/or sustained delivery a triple reuptake inhibitor compound or a pharmaceutically acceptable salt thereof. As used herein, “sustained release” and “sustained delivery” are evinced by a sustained, delayed, extended, or modified, in vitro or in vivo dissolution rate, in vivo release and/or delivery rate, and/or in vivo pharmacokinetic value(s) or profile.

The sustained release dosage forms used in the methods of the invention can take any form as long as one or more of the dissolution, release, delivery and/or pharmacokinetic property(ies) identified above are satisfied. Within illustrative embodiments, the composition or dosage form can comprise a triple reuptake inhibitor compound or a pharmaceutically acceptable salt thereof combined with any one or combination of: a drug-releasing polymer, matrix, bead, microcapsule, or other solid drug-releasing vehicle; drug-releasing tiny timed-release pills or mini-tablets; compressed solid drug delivery vehicle; controlled release binder; multi-layer tablet or other multi-layer or multi-component dosage form; drug-releasing lipid; drug-releasing wax; and a variety of other sustained drug release materials as contemplated herein, or formulated in an osmotic dosage form.

The present invention thus encompasses a broad range of sustained release compositions and dosage forms a triple reuptake inhibitor compound or a pharmaceutically acceptable salt thereof, which in certain embodiments are adapted for providing sustained release of the active compound(s) following, e.g., oral administration. Sustained release vehicles, matrices, binders and coatings for use in accordance with the invention include any biocompatible sustained release material which is inert to the active agent and which is capable of being physically combined, admixed, or incorporated with the active compound. Useful sustained release materials may be dissolved, degraded, disintegrated, and/or metabolized slowly under physiological conditions following delivery (e.g., into a gastrointestinal tract of a subject, or following contact with gastric fluids or other bodily fluids). Useful sustained release materials are typically non-toxic and inert when contacted with fluids and tissues of mammalian subjects, and do not trigger significant adverse side effects such as irritation, immune response, inflammation, or the like. They are typically metabolized into metabolic products which are biocompatible and easily eliminated from the body.

In certain embodiments, sustained release polymeric materials are employed as the sustained release vehicle, matrix, binder, or coating (see, e.g., “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105, each incorporated herein by reference). Within exemplary embodiments, useful polymers for co-formulating with a triple reuptake inhibitor compound or a pharmaceutically acceptable salt thereof to yield a sustained release composition or dosage form include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

Additional polymeric materials for use as sustained release vehicles, matrices, binders, or coatings within the compositions and dosage forms used in the invention include, but are not limited to, additional cellulose ethers, e.g., as described in Alderman, Int. J. Pharm. Tech. & Prod. Mfr., 1984, 5(3) 1-9 (incorporated herein by reference). Other useful polymeric materials and matrices are derived from copolymeric and homopolymeric polyesters having hydrolysable ester linkages. A number of these are known in the art to be biodegradable and to lead to degradation products having no or low toxicity. Exemplary polymers in this context include polyglycolic acids (PGAs) and polylactic acids (PLAs), poly(DL-lactic acid-co-glycolic acid) (DL PLGA), poly(D-lactic acid-coglycolic acid) (D PLGA) and poly(L-lactic acid-co-glycolic acid) (L PLGA). Other biodegradable or bioerodable polymers for use within the invention include such polymers as poly(ε-caprolactone), poly(ε-aprolactone-CO-lactic acid), poly(ε-aprolactone-CO-glycolic acid), poly(β-hydroxy butyric acid), poly(alkyl-2-cyanoacrilate), hydrogels such as poly(hydroxyethyl methacrylate), polyamides, poly-amino acids (e.g., poly-L-leucine, poly-glutamic acid, poly-L-aspartic acid, and the like), poly(ester ureas), poly(2-hydroxyethyl DL-aspartamide), polyacetal polymers, polyorthoesters, polycarbonates, polymaleamides, polysaccharides, and copolymers thereof. Methods for preparing pharmaceutical formulations using these polymeric materials are generally known to those skilled in the art (see, e.g., Sustained and Controlled Release Drug Delivery Systems, J. R. Robinson, ed., Marcel Dekker, Inc., New York, 1978, incorporated herein by reference).

In other embodiments of the invention, the compositions and dosage forms of a triple reuptake inhibitor compound or a pharmaceutically acceptable salt thereof are coated on a polymer substrate. The polymer can be an erodible or a nonerodible polymer. The coated substrate may be folded onto itself to provide a bilayer polymer drug dosage form. For example, a triple reuptake inhibitor compound or a pharmaceutically acceptable salt thereof can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a controlled rate to dispense the active compound over a sustained release period. Representative biodegradable polymers for use in this and other aspects of the invention can be selected from, for example, biodegradable poly(amides), poly(amino acids), poly(esters), poly(lactic acid), poly(glycolic acid), poly(carbohydrate), poly(orthoester), poly(orthocarbonate), poly(acetyl), poly(anhydrides), biodegradable poly(dehydropyrans), and poly(dioxinones) which are known in the art (see, e.g., Rosoff, Controlled Release of Drugs, Chap. 2, pp. 53-95 (1989); and U.S. Pat. Nos. 3,811,444; 3,962,414; 4,066,747, 4,070,347; 4,079,038; and 4,093,709, each incorporated herein by reference).

In another embodiment of the invention, the dosage form comprises a triple reuptake inhibitor compound or a pharmaceutically acceptable salt thereof loaded into a polymer that releases the drug by diffusion through a polymer, or by flux through pores or by rupture of a polymer matrix. The drug delivery polymeric dosage form comprises the active compound contained in or on the polymer. The dosage form comprises at least one exposed surface at the beginning of dose delivery. The non-exposed surface, when present, can be coated with a pharmaceutically acceptable material impermeable to the passage of a drug. The dosage form may be manufactured by procedures known in the art, for example by blending a pharmaceutically acceptable carrier like polyethylene glycol, with a pre-determined dose of the active compound(s) at an elevated temperature (e.g., 37° C.), and adding it to a silastic medical grade elastomer with a cross-linking agent, for example, octanoate, followed by casting in a mold. The step is repeated for each optional successive layer. The system is allowed to set for 1 hour, to provide the dosage form. Representative polymers for manufacturing such sustained release dosage forms include, but are not limited to, olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, poly alginate, polyamide and polysilicon. These polymers and procedures for manufacturing them have been described in the art (see, e.g., Coleman et al., Polymers 1990, 31, 1187-1231; Roerdink et al., Drug Carrier Systems 1989, 9, 57-10; Leong et al., Adv. Drug Delivery Rev. 1987, 1, 199-233; and Roff et al., Handbook of Common Polymers 1971, CRC Press; U.S. Pat. No. 3,992,518).

In other embodiments of the invention, the compositions and dosage forms comprise a triple reuptake inhibitor compound or a pharmaceutically acceptable salt thereof incorporated with or contained in beads that on dissolution or diffusion release the active compound over an extended period of hours, for example over a period of at least 6 hours, over a period of at least 8 hours, over a period of at least 12 hours, or over a period of up to 24 hours or longer. The drug-releasing beads may have a central composition or core comprising a triple reuptake inhibitor compound or a pharmaceutically acceptable salt thereof and a pharmaceutically acceptable carrier, along with one or more optional excipients such as a lubricants, antioxidants, dispersants, and buffers. The beads may be medical preparations with a diameter of about 1 to 2 mm. In exemplary embodiments they are formed of non-cross-linked materials to enhance their discharge from the gastrointestinal tract. The beads may be coated with a release rate-controlling polymer that gives a timed release pharmacokinetic profile. In alternate embodiments the beads may be manufactured into a tablet for therapeutically effective drug administration. The beads can be made into matrix tablets by direct compression of a plurality of beads coated with, for example, an acrylic resin and blended with excipients such as hydroxypropylmethyl cellulose. The manufacture and processing of beads for use within the invention is described in the art (see, e.g., Lu, Int. J. Pharm., 1994, 112, 117-124; Pharmaceutical Sciences by Remington, 14^{th} ed, pp 1626-1628 (1970); Fincher, J. Pharm. Sci. 1968, 57, 1825-1835; and U.S. Pat. No. 4,083,949, each incorporated by reference) as has the manufacture of tablets (Pharmaceutical Sciences, by Remington, 17^{th} Ed, Ch. 90, pp 1603-1625, 1985, incorporated herein by reference).

In another embodiment of the invention, the dosage form comprises a plurality of tiny pills or mini-tablets. The tiny pills or mini-tablets provide a number of individual doses for providing various time doses for achieving a sustained-release drug delivery profile over an extended period of time up to 24 hours. The tiny pills or mini-tablets may comprise a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic polymer may be formed into a plurality (e.g., 4 to 50) tiny pills or mini-tablet, wherein each tiny pill or mini-tablet comprises a pre-determined dose of a triple reuptake inhibitor compound or a pharmaceutically acceptable salt thereof, e.g., a dose of about 10 ng, 0.5 mg, 1 mg, 1.2 mg, 1.4 mg, 1.6 mg, 5.0 mg etc. The tiny pills and mini-tablets may further comprise a release rate-controlling wall of 0.001 up to 10 mm thickness to provide for timed release of the active compound. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills and mini-tablets are known in the art (see, e.g., U.S. Pat. Nos. 4,434,153; 4,721,613; 4,853,229; 2,996,431; 3,139,383 and 4,752,470, each incorporated herein by reference). The tiny pills and mini-tablets may further comprise a blend of particles, which may include particles of different sizes and/or release properties, and the particles may be contained in a hard gelatin or non-gelatin capsule or soft gelatin capsule.

In yet another embodiment of the invention, drug-releasing lipid matrices can be used to formulate therapeutic compositions and dosage forms comprising a triple reuptake inhibitor compound or a pharmaceutically acceptable salt thereof. In one exemplary embodiment, solid microparticles of the active compound are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700 (each incorporated herein by reference). The lipid-coated particles can optionally be compressed to form a tablet. Another controlled release lipid-based matrix material which is suitable for use in the sustained release compositions and dosage forms of the invention comprises polyglycolized glycerides, e.g., as described in Roussin et al., U.S. Pat. No. 6,171,615 (incorporated herein by reference).

In other embodiments of the invention, drug-releasing waxes can be used for producing sustained release compositions and dosage forms comprising a triple reuptake inhibitor or a pharmaceutically acceptable salt thereof. Examples of suitable sustained drug-releasing waxes include, but are not limited to, carnauba wax, candedilla wax, esparto wax, ouricury wax, hydrogenated vegetable oil, bees wax, paraffin, ozokerite, castor wax, and mixtures thereof (see, e.g., Cain et al., U.S. Pat. No. 3,402,240; Shtohryn et al. U.S. Pat. No. 4,820,523; and Walters, U.S. Pat. No. 4,421,736, each incorporated herein by reference).

In still another embodiment, osmotic delivery systems are used for sustained release delivery of a triple reuptake inhibitor compound or a pharmaceutically acceptable salt thereof (see, e.g., Verma et al., Drug Dev. Ind. Pharm., 2000, 26:695-708, incorporated herein by reference). In one exemplary embodiment, the osmotic delivery system is an OROS® system (Alza Corporation, Mountain View, Calif.) and is adapted for oral sustained release delivery of drugs (see, e.g., U.S. Pat. No. 3,845,770; and U.S. Pat. No. 3,916,899, each incorporated herein by reference).

In another embodiment of the invention, the dosage form comprises an osmotic dosage form, which comprises a semi-permeable wall that surrounds a therapeutic composition comprising a triple reuptake inhibitor compound or a pharmaceutically acceptable salt thereof. In use within a patient, the osmotic dosage form comprising a homogenous composition imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained prodrug release. These delivery platforms can provide an essentially zero order delivery profile as opposed to the spiked profiles of immediate release formulations.

In alternate embodiments of the invention, the dosage form comprises another osmotic dosage form comprising a wall surrounding a compartment, the wall comprising a semipermeable polymeric composition permeable to the passage of fluid and substantially impermeable to the passage of the active compound present in the compartment, a drug-containing layer composition in the compartment, a hydrogel push layer composition in the compartment comprising an osmotic formulation for imbibing and absorbing fluid for expanding in size for pushing the triple reuptake inhibitor compound or a pharmaceutically acceptable salt thereof composition layer from the dosage form, and at least one passageway in the wall for releasing the drug composition. This osmotic system delivers the active compound by imbibing fluid through the semipermeable wall at a fluid imbibing rate determined by the permeability of the semipermeable wall and the osmotic pressure across the semipermeable wall causing the push layer to expand, thereby delivering the active compound through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer may comprise a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose), an osmagent, e.g., selected from the group consisting of sodium chloride, potassium chloride, potassium acid phosphate, tartaric acid, citric acid, raffinose, magnesium sulfate, magnesium chloride, urea, inositol, sucrose, glucose and sorbitol, and other agents such a hydroxypropylalkylcellulose of 9,000 to 225,000 average-number molecular weight (e.g., hydroxypropylethylcellulose, hydroxypropypentylcellulose, hydroxypropylmethylcellulose, or hydropropylbutylcellulose), ferric oxide, antioxidants (e.g., ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine), and/or lubricants (e.g., calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid).

In the osmotic dosage forms, the semipermeable wall comprises a composition that is permeable to the passage of fluid and impermeable to passage of the triple reuptake inhibitor compound or a pharmaceutically acceptable salt thereof. The wall is nontoxic and comprises a polymer selected from the group consisting of a cellulose acylate, cellulose diacylate, cellulose triacylate, cellulose acetate, cellulose diacetate and cellulose triacetate. The wall typically comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether (e.g., hydroxypropylcellulose or a hydroxypropylalkycellulose such as hydroxypropylmethylcellulose). The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of drug to a patient over time. The dosage form comprises a passageway in the wall that connects the exterior of the dosage form with the internal compartment. The osmotic powered dosage form delivers the triple reuptake inhibitor compound or a pharmaceutically acceptable salt thereof from the dosage form to the patient at a zero order rate of release over a period of up to about 24 hours. As used herein, the expression “passageway” comprises means and methods suitable for the metered release of a triple reuptake inhibitor compound or a pharmaceutically acceptable salt thereof from the compartment of an osmotic dosage form. The exit means comprises at least one passageway, including orifice, bore, aperture, pore, porous element, hollow fiber, capillary tube, channel, porous overlay, or porous element that provides for the osmotic controlled release of the active compound. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864; 4,816,263; 4,200,098; and 4,285,987 (each incorporated herein by reference).

In more detailed embodiments, a compound as disclosed herein may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

A variety of methods is known by which a triple reuptake inhibitor compound or a pharmaceutically acceptable salt thereof can be encapsulated in the form of microparticles, for example using by encapsulating the active compound within a biocompatible, biodegradable wall-forming material (e.g., a polymer)—to provide sustained or delayed release of the active compound. In these methods, the active compound is typically dissolved, dispersed, or emulsified in a solvent containing the wall forming material. Solvent is then removed from the microparticles to form the finished microparticle product. Examples of conventional microencapsulation processes are disclosed, e.g., in U.S. Pat. Nos. 3,737,337; 4,389,330; 4,652,441; 4,917,893; 4,677,191; 4,728,721; 5,407,609; 5,650,173; 5,654,008; and 6,544,559 (each incorporated herein by reference). These documents disclose methods that can be readily implemented to prepare microparticles containing a triple reuptake inhibitor compound or a pharmaceutically acceptable salt thereof in a sustained release formulation according to the invention. As explained, for example, in U.S. Pat. No. 5,650,173, by appropriately selecting the polymeric materials, a microparticle formulation can be made in which the resulting microparticles exhibit both diffusional release and biodegradation release properties. For a diffusional mechanism of release, the active agent is released from the microparticles prior to substantial degradation of the polymer. The active agent can also be released from the microparticles as the polymeric excipient erodes. In addition, U.S. Pat. No. 6,596,316 (incorporated herein by reference) discloses methods for preparing microparticles having a selected release profile for fine tuning a release profile of an active agent from the microparticles.

In another embodiment of the invention, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of a triple reuptake inhibitor compound or a pharmaceutically acceptable salt thereof by providing one or more barrier layers, which may be located entirely surrounding the active compound, between layers of a multi-layer solid dosage form (see below), and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Barrier layers comprising polymers which swell in contact with water or aqueous media may swell to such an extent that the swollen layer forms a relatively large swollen mass, the size of which delays its immediate discharge from the stomach into the intestine. The barrier layer may itself contain active material content, for example the barrier layer may be a slow or delayed release layer. Barrier layers may typically have an individual thickness of 10 microns up to 2 mm. Suitable polymers for barrier layers which are relatively impermeable to water include the Methocel™ series of polymers, used singly or combined, and Ethocel™ polymers. Such polymers may suitably be used in combination with a plasticizer such as hydrogenated castor oil. The barrier layer may also include conventional binders, fillers, lubricants and compression acids etc. such as Polyvidon K30 (trade mark), magnesium stearate, and silicon dioxide.

Additional enteric coating materials for mediating sustained release of a triple reuptake inhibitor or a pharmaceutically acceptable salt thereof include coatings in the form of polymeric membranes, which may be semipermeable, porous, or asymmetric membranes (see, e.g., U.S. Pat. No. 6,706,283, incorporated herein by reference). Coatings of these and other types for use within the invention may also comprise at least one delivery port, or pores, in the coating, e.g., formed by laser drilling or erosion of a plug of water-soluble material. Other useful coatings within the invention including coatings that rupture in an environment of use (e.g., a gastrointestinal compartment) to form a site of release or delivery port. Exemplary coatings within these and other embodiments of the invention include poly(acrylic) acids and esters; poly(methacrylic) acids and esters; copolymers of poly(acrylic) and poly(methacrylic) acids and esters; cellulose esters; cellulose ethers; and cellulose ester/ethers.

Additional coating materials for use in constructing solid dosage forms to mediate sustained release of a triple reuptake inhibitor compound or a pharmaceutically acceptable salt thereof include, but are not limited to, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethyl-cellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxlated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

In additional embodiments of the invention, sustained release of a triple reuptake inhibitor compound or a pharmaceutically acceptable salt thereof is provided by formulating the active compound in a dosage form comprising a multi-layer tablet or other multi-layer or multi-component dosage form. In exemplary embodiments, the active compound is formulated in layered tablets, for example having a first layer which is an immediate release layer and a second layer which is a slow release layer. Other multi-layered dosage forms of the invention may comprise a plurality of layers of compressed active ingredient having variable (i.e., selectable) release properties selected from immediate, extended and/or delayed release mechanisms. Multi-layered tablet technologies useful to produce sustained release dosage forms of a triple reuptake inhibitor compound or a pharmaceutically acceptable salt thereof are described, for example, in International Publications WO 95/20946; WO 94/06416; and WO 98/05305 (each incorporated herein by reference). Other multi-component dosage forms for providing sustained delivery of a triple reuptake inhibitor compound or a pharmaceutically acceptable salt thereof include tablet formulations having a core containing the active compound coated with a release retarding agent and surrounded by an outer casing layer (optionally containing the active compound) (see, e.g., International Publication WO 95/28148, incorporated herein by reference). The release retarding agent is an enteric coating, so that there is an immediate release of the contents of the outer core, followed by a second phase from the core which is delayed until the core reaches the intestine. Additionally, International Publication WO 96/04908 (incorporated herein by reference) describes tablet formulations which comprise an active agent in a matrix, for immediate release, and granules in a delayed release form comprising the active agent. Such granules are coated with an enteric coating, so release is delayed until the granules reach the intestine. International Publication WO 96/04908 (incorporated herein by reference) describes delayed or sustained release formulations formed from granules which have a core comprising an active agent, surrounded by a layer comprising the active agent.

Another useful multi-component (bi-layer tablet) dosage form for sustained delivery of a triple reuptake inhibitor compound or a pharmaceutically acceptable salt thereof is described in U.S. Pat. No. 6,878,386 (incorporated herein by reference). Briefly, the bilayer tablet comprises an immediate release and a slow release layer, optionally with a coating layer. The immediate release layer may be, for example, a layer which disintegrates immediately or rapidly and has a composition similar to that of known tablets which disintegrate immediately or rapidly. An alternative type of immediate release layer may be a swellable layer having a composition which incorporates polymeric materials which swell immediately and extensively in contact with water or aqueous media, to form a water permeable but relatively large swollen mass. Active material content may be immediately leached out of this mass. The slow release layer may have a composition comprising a triple reuptake inhibitor compound or a pharmaceutically acceptable salt thereof with a release retarding vehicle, matrix, binder, coating, or excipient which allows for slow release of the active compound. Suitable release retarding excipients include pH sensitive polymers, for instance polymers based upon methacrylic acid copolymers, which may be used either alone or with a plasticiser; release-retarding polymers which have a high degree of swelling in contact with water or aqueous media such as the stomach contents; polymeric materials which form a gel on contact with water or aqueous media; and polymeric materials which have both swelling and gelling characteristics in contact with water or aqueous media. Release retarding polymers which have a high degree of swelling include, inter alia, cross-linked sodium carboxymethylcellulose, cross-linked hydroxypropylcellulose, high-molecular weight hydroxypropylmethylcellulose, carboxymethylamide, potassium methacrylatedivinylbenzene co-polymer, polymethylmethacrylate, cross-linked polyvinylpyrrolidone, high-molecular weight polyvinylalcohols etc. Release retarding gellable polymers include methylcellulose, carboxymethylcellulose, low-molecular weight hydroxypropylmethylcellulose, low-molecular weight polyvinylalcohols, polyoxyethyleneglycols, non-cross linked polyvinylpyrrolidone, xanthan gum etc. Release retarding polymers simultaneously possessing swelling and gelling properties include medium-viscosity hydroxypropylmethylcellulose and medium-viscosity polyvinylalcohols. An exemplary release-retarding polymer is xanthan gum, in particular a fine mesh grade of xanthan gum, preferably pharmaceutical grade xanthan gum, 200 mesh, for instance the product Xantural 75 (also known as Keltrol CR™ Monsanto, 800 N Lindbergh Blvd, St Louis, Mo. 63167, USA). Xanthan gum is a polysaccharide which upon hydration forms a viscous gel layer around the tablet through which the active has to diffuse. It has been shown that the smaller the particle size, the slower the release rate. In addition, the rate of release of active compound is dependent upon the amount of xanthan gum used and can be adjusted to give the desired profile. Examples of other polymers which may be used within these aspects of the invention include Methocel K4M™, Methocel E5™, Methocel E50™, Methocel E4M™, Methocel K15M™ and Methocel K100M™. Other known release-retarding polymers which may be incorporated within this and other embodiments of the invention to provide a sustained release composition or dosage form of a triple reuptake inhibitor compound or a pharmaceutically acceptable salt thereof include, hydrocolloids such as natural or synthetic gums, cellulose derivatives other than those listed above, carbohydrate-based substances such as acacia, gum tragacanth, locust bean gum, guar gum, agar, pectin, carrageenan, soluble and insoluble alginates, carboxypolymethylene, casein, zein, and the like, and proteinaceous substances such as gelatin.

Within other embodiments of the invention, a sustained release delivery device or system is placed in the subject in proximity of the target of the active compound, thus requiring only a fraction of the systemic dose (see, e.g., Goodson, in “Medical Applications of Controlled Release,” supra, vol. 2, pp. 115-138, 1984; and Langer, 1990, Science 249:1527-1533, each incorporated herein by reference). In other embodiments, an oral sustained release pump may be used (see, e.g., Langer, supra; Sefton, 1987, CRC Crit. Ref. Biomed. Eng. 14:201; and Saudek et al., 1989, N. Engl. J. Med. 321:574, each incorporated herein by reference).

The pharmaceutical compositions and dosage forms used in the current invention will typically be provided for administration in a sterile or readily sterilizable, biologically inert, and easily administered form.

In other embodiments the invention provides pharmaceutical kits for reducing symptoms in a human subject suffering from a disorder affected by monoamine neurotransmitters, including depression. The kits comprise a triple reuptake inhibitor compound or a pharmaceutically acceptable salt thereof in an effective amount, and a container means for containing the triple reuptake inhibitor compound or a pharmaceutically acceptable salt thereof for coordinate administration to the said subject (for example a container, divided bottle, or divided foil pack). The container means can include a package bearing a label or insert that provides instructions for multiple uses of the kit contents to treat the disorder and reduce symptoms in the subject. In more detailed embodiments, the triple reuptake inhibitor compound or a pharmaceutically acceptable salt thereof is admixed or co-formulated in a single, combined dosage form, for example a liquid or solid oral dosage form. In alternate embodiments, the triple reuptake inhibitor compound or a pharmaceutically acceptable salt thereof is contained in the kit in separate dosage forms for coordinate administration. An example of such a kit is a so-called blister pack. Blister packs are well-known in the packaging industry and are widely used for the packaging of pharmaceutical dosage forms (tablets, capsules and the like).

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The following examples illustrate certain aspects of the invention, but are not intended to limit in any manner the scope of the invention.

### Example I

### Efficacy of (+)-1-(3,4-dichlorophenyl)-3-azabicyclo[3.1.0]hexane in the Treatment of Patients with Major Depressive Disorder

Subjects were identified who were between the ages of 18-65 (inclusive), and met criteria for Major Depressive Disorder in accordance with the Diagnostic and Statistical manual of Mental Disorders-IV-TR and confirmed by the MINI International Neuropsychiatric Interview. At the screening visit, subjects had a baseline Hamilton Depression Rating Scale (HAMD-17)≧22 and a severity of ≧2 on item 1 and a rating on the Hamilton Anxiety Scale (HAM-A)<17. They were also required to have a BMI≦35 and body weight >45 kg at the Screening Visit.

They were excluded if they were judged to be a suicide risk, known to be antidepressant treatment resistant or had other major clinically significant medical and/or other psychiatric illnesses such as panic disorder, social phobia, generalized anxiety disorder, obsessive compulsive disorder, post-traumatic stress disorder, acute stress disorder, substance abuse, anorexia, bulimia, antisocial personality disorder or bipolar disorder. Additionally, subjects who had a HAMD-17 reduction in score of more than 15% between the Placebo run-in visit and the baseline visit were eliminated.

Subjects were required to refrain from taking antidepressants, anticonvulsants including gabapentin and pregabalin, neuroleptics, MAO inhibitors, barbiturates, benzodiazepines, stimulants, antipsychotics, lithium, anxiolytics and beta blockers starting two weeks prior to the study and continuing until after the follow-up visit.

Subjects were evaluated for safety parameters prior to and throughout the trial by a variety of measures including electrocardiogram, physical examination, vital signs and body weight, and clinical laboratory testing including a lipid panel, CBC with differential and urinalysis. Sixty-three eligible subjects were identified who were not eliminated by the safety parameters. These sixty-three subjects had the following combined (placebo and (+)-1-(3,4-dichlorophenyl)-3-azabicyclo[3.1.0]hexane) mean baseline scores on the main outcome measures: MADRS (31.4) (primary); HAMD-17 (29.6) (secondary); and DISF-SR (25.38). The sixty-three subjects were randomized to receive either 25 mg of (+)-1-(3,4-dichlorophenyl)-3-azabicyclo[3.1.0]hexane twice a day for two weeks and then 50 mg twice a day for four weeks or placebo.

Efficacy was determined by measuring the change from baseline in the Montgomery-Åsberg Depression Rating Scale (MADRS), the HAMD-17, the Clinical Global Impression Global Improvement Scale (CGI-I), the Clinical Global Impression-Severity scale (CGI-S) and the Derogatis Interview for Sexual Functioning Self-Report (DISF-SR). Two analysis populations were studied: Modified Intent to Treat (MITT, N=56), defined as all randomized subjects with any confirmed dosing and MADRS data from at least one post-baseline visit (30 (+)-1-(3,4-dichlorophenyl)-3-azabicyclo[3.1.0]hexane-treated patients and 26 placebo-treated patients); and Completers (N=39), defined as the subset of MITT subjects who completed 6 weeks of treatment (20 (+)-1-(3,4-dichlorophenyl)-3-azabicyclo[3.1.0]hexane-treated patients and 19 placebo-treated patients). Comparisons between treatment groups based on MADRS (the primary efficacy parameter), HAMD-17, Anhedonia, DISF-SR, CGI-I and CGI-S scores were analyzed using a mixed-repeated measures (MMRM) analysis model including factors for Subject, Visit, Treatment Arm and Baseline value as a covariate. Adjusted least-squares means from these models are presented. Comparisons between groups were made at each post-baseline visit using model-based contrasts and adjusted degrees of freedom. For these analyses no explicit data imputations were made prior to the analysis. Response and remission categorical data were analyzed using chi-square tests. Inferential analyses of safety data were conducted with ANOVA models or chi-square tests. Two-tail alpha was set to 0.05. All analyses were conducted using SAS version 9.2.

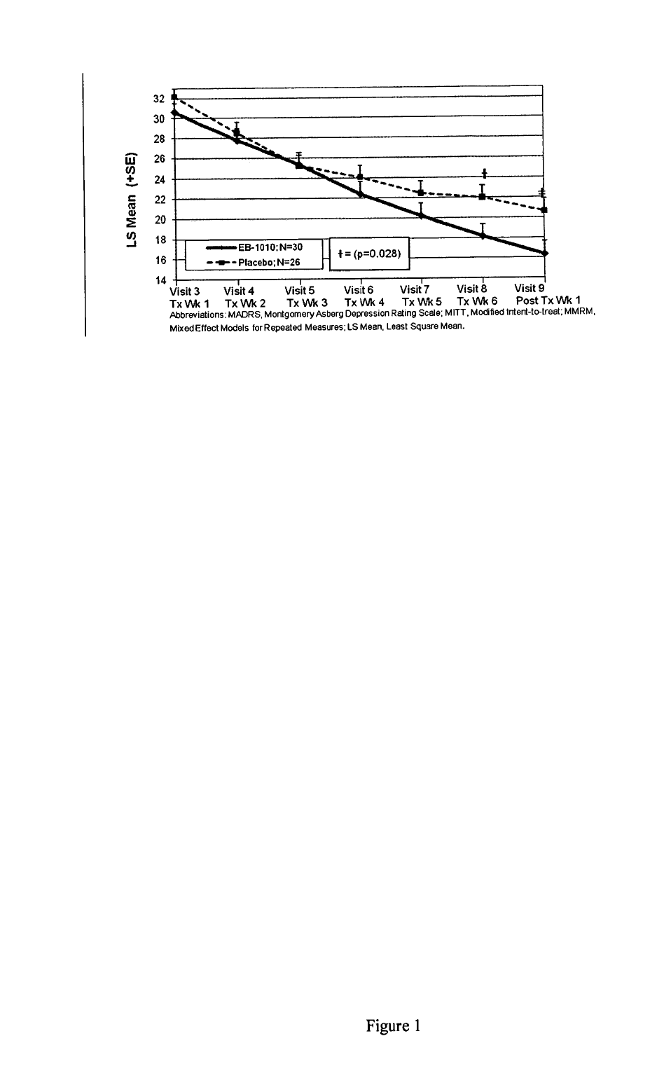

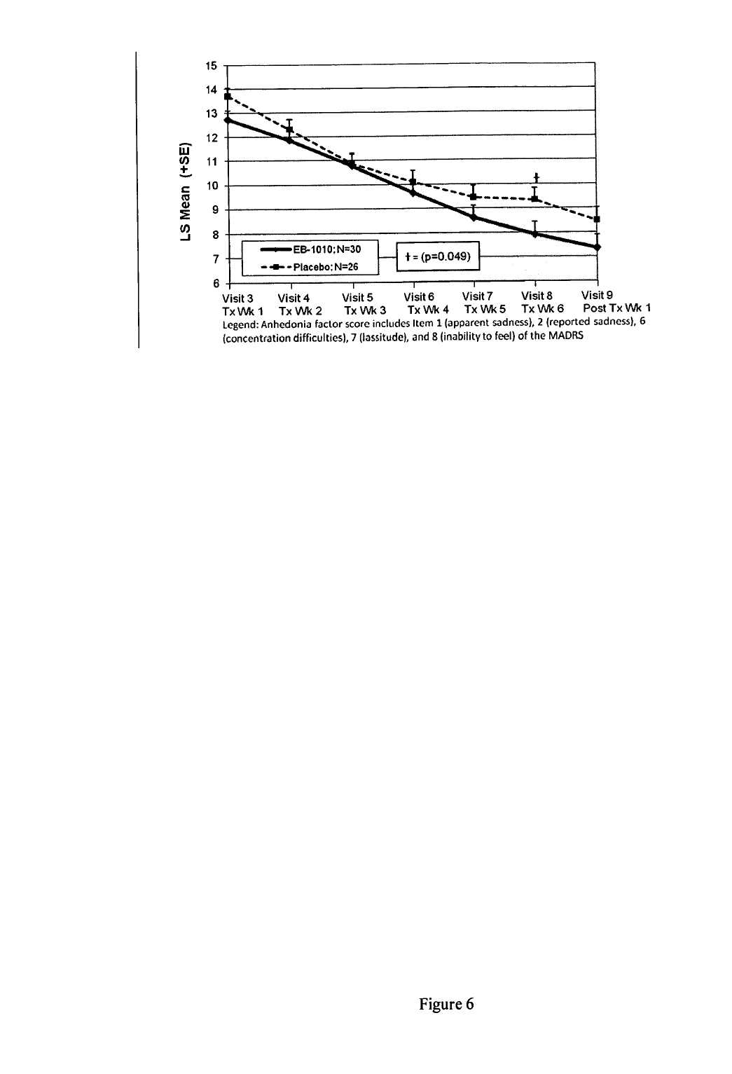

The intent-to-treat (ITT) population (n=56) showed the following combined (placebo and (+)-1-(3,4-dichlorophenyl)-3-azabicyclo[3.1.0]hexane) mean baseline scores on the main outcome measures: MADRS (31.4) (primary); HAMD-17 (29.5) (secondary); and DISF-SR (25.8). As shown in FIG. 1, at the end of the double-blind treatment (Week 6), the estimated LS mean change from baseline (MMRM or mixed model repeated measures) in the MADRS total scores was statistically significantly superior for (+)-1-(3,4-dichlorophenyl)-3-azabicyclo[3.1.0]hexane when compared to placebo (18.16 vs 21.99; p=0.028), with an overall statistical effect size of −0.63 (Cohen's d). As shown in Table 1, when assessed with the CGI-I, a global impression scale sensitive to clinically relevant changes in improvement status, treatment with (+)-1-(3,4-dichlorophenyl)-3-azabicyclo[3.1.0]hexane was also statistically significantly superior to placebo (p=0.03; Week 6; MMRM). As shown in FIG. 6, an anhedonia factor score grouping Items 1 (apparent sadness), 2 (reported sadness), 6 (concentration difficulties), 7 (lassitude), and 8 (inability to feel) of the MADRS (analyzed using the mixed model for repeated measures LS means) demonstrated a statistically significant difference in favor of (+)-1-(3,4-dichlorophenyl)-3-azabicyclo[3.1.0]hexane in comparison to placebo (p=0.049). (+)-1-(3,4-dichlorophenyl)-3-azabicyclo[3.1.0]hexane was relatively well tolerated. Two patients in each treatment group discontinued the study early due to AEs but no serious AEs were reported.

TABLE 1
Least Square Adjusted Means with differences in Primary and Secondary Efficacy
Measures at Visit 8 (MMRM, MITT)

| | | (+)-1-(3,4- | | |
|---|---|---|---|---|
| | | dichlorophenyl)-3- | | |
| | | azabicyclo[3.1.0]hexane | Difference (95% | |
| Outcome | Placebo (n = 26) | (n = 30) | CI) | P value |
| MADRS (LS Mean-SE) | 21.99 (1.24) | 18.16 (1.21) | 3.83 (0.41, 7.26) | P = 0.028 |
| HAMD-17 (LS Mean-SE) | 18.02 (1.46) | 14.90 (1.40) | 3.12 (−0.87, 7.12) | P = 0.125 |
| Anhedonia factor (LS Mean- | 9.33 (0.50) | 7.92 (0.50) | 1.41 (0.01, 2.82) | P = 0.049 |
| SE) | | | | |
| CGI-I (LS Mean-SE) | 2.75 (0.20) | 2.13 (0.20) | 0.62 (0.06, 1.18) | P = 0.030 |
| CGI-S (LS Mean-SE) | 3.53 (0.15) | 3.31 (0.15) | 0.22 (−0.21, 0.66) | P = 0.306 |

Abbreviations:
MADRS, Montgomery Åsberg Depression Rating Scale;
HAMD-17, Hamilton Rating Scale for Depression;
CGI-I, Clinical Global Impressions - Improvement;
CGI-S, Clinical Global Impressions - Severity;
MMRM, Mixed Effect Models for Repeated Measures;
MITT, Modified Intent-to-treat;
CI, Confidence Interval,
SE, Standard Error.

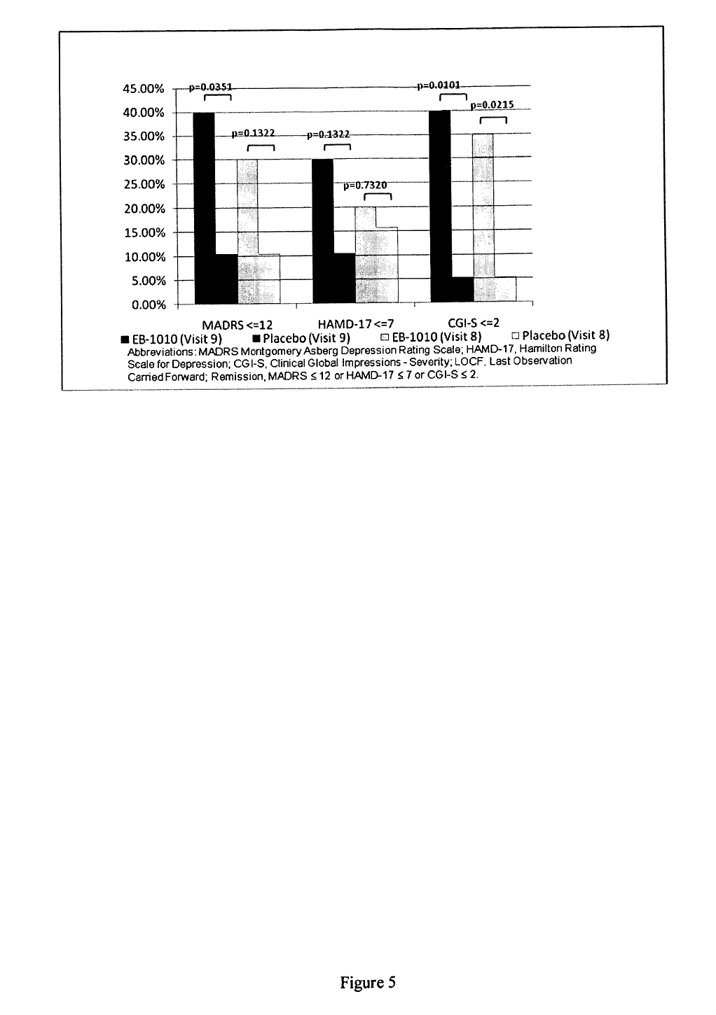

As shown in Table 2 and FIG. 5 (data analyzed using the last observation carried forward method), treatment with 100 mg of (+)-1-(3,4-dichlorophenyl)-3-azabicyclo[3.1.0]hexane was associated with significantly greater remission rates, defined by achieving a CGI-S score of ≦2, compared to placebo.

TABLE 2
Response and Remission Rates (Visit 8, LOCF, Completers)

| | (+)-1-(3,4-dichlorophenyl)- | | | |
|---|---|---|---|---|
| | 3-azabicyclo[3.1.0]hexane | | | |
| Outcome | 100 mg [n/N] (%) | Placebo [n/N] (%) | Odds Ratio (95% CI) | P value |
| Response | | | | |
| MADRS | (8/20) 40.00% | (3/19) 15.79% | 0.281 (0.061, 1.290) | 0.093 |
| HAMD-17 | (11/20) 55.00% | (7/19) 36.84% | 0.477 (0.132, 1.721) | 0.256 |
| Remission | | | | |
| MADRS | (6/20) 30.00% | (2/19) 10.53% | 0.275 (0.048, 1.579) | 0.132 |
| HAMD-17 | (4/20) 20.00% | (3/19) 15.79% | 0.750 (0.144, 3.904) | 0.732 |
| CGI-S | (7/20) 35.00% | (1/19) 5.26% | 0.103 (0.011, 0.944) | 0.022 |

Abbreviations:
MADRS, Montgomery Åsberg Depression Rating Scale;
HAMD-17, Hamilton Rating Scale for Depression;
CGI-I, Clinical Global Impressions - Improvement;
LOCF, Last Observation Carried Forward;
Response, 50% reduction or more of the baseline total score of MADRS or HAMD-17 at endpoint;
Remission, MADRS ≦12 or HAMD-17 ≦7 or CGI-S ≦2.

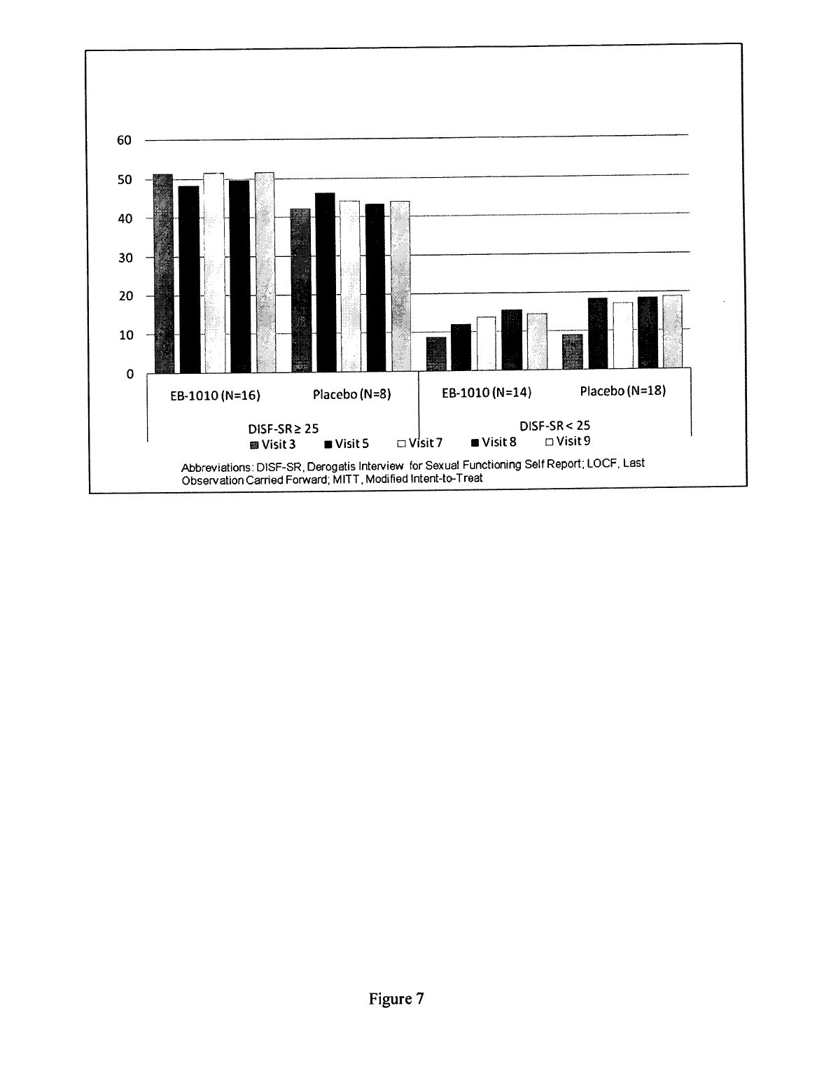

Additionally, unlike many antidepressants, as shown in FIG. 7, the DISF-SR scores stratified by low mean baseline scores (<25, indicating poor sexual function at baseline) versus high mean baseline scores (≧25, indicating preserved sexual function at baseline). In both the low baseline and high baseline groups, there are no differences between (+)-1-(3,4-dichlorophenyl)-3-azabicyclo[3.1.0]hexane 100 mg and placebo, indicating that treatment with (+)-1-(3,4-dichlorophenyl)-3-azabicyclo[3.1.0]hexane is not associated with emergence of sexual dysfunction. The efficacy of treatment with (+)-1-(3,4-dichlorophenyl)-3-azabicyclo[3.1.0]hexane was observed on the primary and secondary standard validated depression outcome measures (MADRS; global severity and improvement) as well as on the anhedonia factor of the MADRS. Furthermore, treatment with (+)-1-(3,4-dichlorophenyl)-3-azabicyclo[3.1.0]hexane was well tolerated and did not result in significant increases in heart rate, systolic or diastolic blood pressure compared to placebo. The number and percentage of patients who reported an adverse treatment event was similar between the two treatment groups (10 or 30.30% for (+)-1-(3,4-dichlorophenyl)-3-azabicyclo[3.1.0]hexane versus 11 or 39.28% for placebo). Additionally, treatment with (+)-1-(3,4-dichlorophenyl)-3-azabicyclo[3.1.0]hexane was not associated with significant weight gain or sexual dysfunction (See, for example, FIG. 7).

The results of this Phase 2 study demonstrated that (+)-1-(3,4-dichlorophenyl)-3-azabicyclo[3.1.0]hexane, at a titrated dose of 50 mg/day then 100 mg/day, was effective for treatment of patients with MDD. Efficacy was observed on the primary and secondary standard validated depression outcome measures (MADRS; global severity and improvement) as well as on the anhedonia factor of the MADRS. Overall, treatment with (+)-1-(3,4-dichlorophenyl)-3-azabicyclo[3.1.0]hexane was well tolerated. The discontinuation rate due to AE was similar to placebo and treatment with (+)-1-(3,4-dichlorophenyl)-3-azabicyclo[3.1.0]hexane was not associated with weight gain or sexual dysfunction.

### Example II

### Occupancy Level of Serotonin Transporters in the Brain Following Administration of (+)-1-(3,4-dichlorophenyl)-3-azabicyclo[3.1.0]hexane

The level of occupancy of serotonin transporters (SERT) in the human brain following administration of (+)-1-(3,4-dichlorophenyl)-3-azabicyclo[3.1.0]hexane was determined in a clinical study. This study was a Phase 1, single-dose, randomized, open-label study using positron emission tomography (PET) and [¹¹C]DASB ([¹¹C]N,N-dimethyl-2-(2-amino-4-cyanophenylthio)benzylamine) as a PET tracer in 3 healthy, young, adult male volunteer subjects. Using PET imaging, uptake inhibitor effects may be measured based on the proportion of SERT sites blocked in the brain.

The subjects were administered a single oral dose of 150 mg (+)-1-(3,4-dichlorophenyl)-3-azabicyclo[3.1.0]hexane. PET scans were done at baseline and at 2 and 7-hour post-dose via measurement of [¹¹C]DASB tracer binding. Periodic blood samples were collected for evaluation of (+)-1-(3,4-dichlorophenyl)-3-azabicyclo[3.1.0]hexane PK. Safety was monitored by clinical and laboratory evaluations, including vital signs, physical examination, clinical chemistry, hematology, urinalysis, and ECG.

A summary of the results is provided in Table 3. At approximately 2 hours after dosing, the mean SERT occupancy was approximately 48%. At approximately 7 hours after dosing, the mean SERT occupancy was approximately 32%. No serious adverse events were reported, and no subjects were discontinued from the study due to a clinical or laboratory adverse experience. One subject reported a total of 5 adverse events, all of which were rated by the investigator as definitely not related to study drug. Other safety evaluations, such as physical examinations and ECGs, revealed no clinically meaningful changes from pre-dose evaluations. No dose-related changes in vital sign measurements (semi-recumbent blood pressure, pulse rate, respiratory rate, and oral temperature) were noted. There were no clinically meaningful changes in the laboratory safety tests.

TABLE 3
Preliminary Mean Plasma Concentration and Mean SERT
Occupancy at about 2 and 7 hours after Administration of 150 mg
(+)-1-(3,4-dichlorophenyl)-3-azabicyclo[3.1.0]hexane

| | Mean (Range) | Mean (Range) | | |
|---|---|---|---|---|
| PET Scan | Plasma Concentration | SERT Occupancy (%) (N = 3) | | |
| Time | (ng/ml) | Thalamus | Striatum | Mean |
| ~2 h | 1107 (1044-1192) | 48 (41-55) | 48 (44-53) | 48 (43-54) |
| ~7 h | 405 (361-483) | 33 (22-44) | 32 (23-37) | 32 (23-41) |

It was concluded that 1) brain SERT occupancy after administration of a single oral dose of 150 mg (+)-1-(3,4-dichlorophenyl)-3-azabicyclo[3.1.0]hexane, as assessed by PET at approximately 2 and 7 hours after dosing, is approximately 48% and 32%, respectively; and 2) mean plasma concentrations of (+)-1-(3,4-dichlorophenyl)-3-azabicyclo[3.1.0]hexane at 2 and 7 hours after administration of a single oral dose 150 mg is approximately 1,107 ng/mL and 405 ng/mL, respectively.

### Example III

### Efficacy of (1R,5S)-(+)-1-(naphthalen-2-yl)-3-azabicyclo[3.1.0]hexane in the Treatment of Adults with ADHD

The efficacy of (1R,5S)-(+)-1-(naphthalen-2-yl)-3-azabicyclo[3.1.0]hexane in treating adult subjects for ADHD is assessed in a clinical study, similar to that described by Spencer et al., 1998. The study consists of a randomized, double-blind, placebo-controlled, crossover study of (1R,5S)-(+)-1-(naphthalen-2-yl)-3-azabicyclo[3.1.0]hexane in the treatment of adults with ADHD.

Subjects between the ages of 19-60 years of age meet DSM-IV-TR criteria for ADHD, describe a chronic course of ADHD symptoms, and endorse impairment associated with ADHD. Criteria excluding potential subjects include clinically significant chronic medical conditions, abnormal baseline laboratory values, psychiatric disorders, drug or alcohol abuse, current use or use in the previous 3 months of psychotropic medication, and mental retardation.

The study design includes two four-week treatment periods separated by a two-week washout period. Study medication is administered at 100 mg/day (50 mg b.i.d.) in an oral formulation, either tablet or capsule. Subjects are seen and evaluated each week over the four-week treatment period. Prior to and throughout the trial, subjects are evaluated for safety parameters by a variety of measures including assessing blood pressure, heart rate, weight, medication accountability and tolerability, and adverse effects.

Efficacy is determined by measuring the change from the baseline of an ADHD rating scale, such as the ADHD Rating Scale or the Conners Adult ADHD Rating Scale (CAARS), which can be investigator rated or self rated. Efficacy in treating ADHD or improvement in ADHD is defined as a reduction in the rating scale score of approximately 30% or more at the endpoint of treatment, and a reduction that is at least 10%, preferably 15-20% greater than that observed with placebo. The statistical significance of results is analyzed using statistical methods known in the art.

All publications and patents cited herein are incorporated herein by reference for the purpose of describing and disclosing, for example, the materials and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

Beck, A. T., R. A Steer (1988) Manual for Beck Hopelessness Scale. Psychological Corp., Harcourt Brace Jovanovich San Antonio, Tex.
Beck, A. T., R. A Steer (1991) Manual for Beck Scale For Suicide Ideation. San Antonio, Tex.: Psychological Corporation.
Bymaster F P, Katner J S, Nelson D L, Hemrick-Luecke S K, Threlkeld P G, Heiligenstein J H, Morin S M, Gehlert D R, Perry K W (2002a) Atomoxetine increases extracellular levels of norepinephrine and dopamine in prefrontal cortex of rat: a potential mechanism for efficacy in attention deficit/hyperactivity disorder. Neuropsychopharmacology 27:699-711.
Bymaster F P, Zhang W, Carter P A, Shaw J, Chemet E, Phebus L, Wong D T, Perry K W (2002b). Fluoxetine, but not other selective serotonin uptake inhibitors, increases norepinephrine and dopamine extracellular levels in prefrontal cortex. Psychopharmacology 160:353-61.
Carboni E, Tanda G L, Frau R, Di Chiara G (1990) Blockade of the noradrenaline carrier increases extracellular dopamine concentrations in the prefrontal cortex: evidence that dopamine is taken up in vivo by noradrenergic terminals. J Neurochem 55:1067-70.
Chandler G M, Iosifescu D V, Pollack M H, Targum S D, Fava M Validation of the Massachusetts General Hospital Antidepressant Treatment History Questionnaire (ATRQ) CNS Neurosci Ther. 2009 Sep. 21. [Epub ahead of print]
Conners, C. K., Erhardt, D. and Sparrow, E., Conners' adult ADHD rating scales, Technical Manual: Multi-Health Systems, New York, USA (1999).
Coric, Vladimir•Stock, Elyse G•Pultz, Joseph•Marcus, Ronald•Sheehan, David V. Sheehan Suicidality Tracking Scale (Sheehan-STS): Preliminary Results from a Multicenter Clinical Trial in Generalized Anxiety Disorder. Psychiatry (Edgmont (Pa.: Township)) 2009 6 (1): 26-31.
Daws L C (2009) Unfaithful neurotransmitter transporters: focus on serotonin uptake and implications for antidepressant efficacy. Pharmacol Ther 121(1):89-99.
Delaville C, De Deurwaerdere P, and Benazzouz, A (2011) Noradrenaline and Parkinson's disease. Frontiers in Systems Neurosci. 5:1-12.
Du Paul G J, Power T J, Anastopoulos A D and Reid R, ADHD rating scale-IV: Checklists norms and clinical interpretation, Guildford Press, New York (1998).
First, Michael B., Williams, Janet B. W., Spitzer, Robert L., and Gibbon, Miriam: Structured Clinical Interview for DSM-IV-T R Axis I Disorders, Clinical Trials Version (SCID-CT). New York: Biometrics Research, New York State Psychiatric Institute, 2007.
Fuxe K, Dahlström A, Höistad M, Marcellino D, Jansson A, Rivera A, Diaz-Cabiale Z, Jacobsen K, Tinner-Staines B, Hagman B, Leo G, Staines W, Guidolin D, Kehr J, Genedani S, Belluardo N, Agnati L F (2007) From the Golgi-Cajal mapping to the transmitter-based characterization of the neuronal networks leading to two modes of brain communication: wiring and volume transmission. Brain Res Rev 55:17-54.
Guy W. Clinician Global Impression (CGI). ECDEU Assessment Manual for Psychopharmacology. 1976. Rockville, Md., U. S. Department of Health, Education, and Welfare.
Hamilton M. A rating scale for depression. J. Neurol. Neurosurg. Psychiat., 1960, 23, 56.
Hamilton M. Development of a rating scale for primary depressive illness. Br J Soc Clin Psychol. 1967; 6(4):278-96
Hamilton, M. (1959). The assessment of anxiety states by rating. British Journal of Medical Psychology, 32, 50-55.
Iyengar S, Webster A A, Hemrick-Luecke S K, Xu J Y, and Simmons R M A (2004) Efficacy of Duloxetine, a Potent and Balanced Serotonin-Norepinephrine Reuptake Inhibitor in Persistent Pain Models in Rats. J. of Pharm. And Experimental Therapeutics, 311: 576-584.
Jones C K, Peters S C and Harlan E S (2005) Efficacy of Duloxetine, a Potent and Balance Serotonergic and Noradrenergic Reuptake Inhibitor, in Inflammatory and Acute Pain Models in Rodents. J. of Pharm. And Experimental Therapeutics, 312: 726-732.
Montgomery S A, Åsberg M. A new depression scale designed to be sensitive to change. Br Psychiatry 1979; 134:382-9
Montgomery, S. A. & Åsberg, M. (1979) A New Depression Scale Designed To Be Sensitive To Change. British Journal of Psychiatry. Vol. 134, pp. 382-389.
Olsen, L. R., et al., The internal and external validity of the Major Depression Inventory in measuring severity of depressive states Psychological Medicine (2003), 33: 351-356 Cambridge University Press.
Papakostas G I, Fava M, Thase M E. Treatment of SSRI-resistant depression: a meta-analysis comparing within-versus across-class switches. Biol Psychiatry. 2008 Apr. 1; 63(7):699-704
Posner K, Oquendo M, Gould M, et al. Columbia Classification Algorithm of Suicide Assessment (C CASA): classification of suicidal events in the FDA's pediatric suicidal risk analysis of antidepressants. Am J Psychiatry. 2007; 165:1035-1043.
Quick Reference to the Diagnostic Criteria From DSM-IV (Diagnostic and Statistical Manual of Mental Disorders, Fourth Edition), The American Psychiatric Association, Washington, D.C., 1994 [0457] Perovic, S, and Muller, W. E., Arzneimittelforschung 45: 1145-1148 (1995)
Sheehan D V, Y Lecrubier, K H Sheehan, P Amorim, J Janavs, E Weiller, T Hergueta, R Baker, G C Dunbar. The Mini-International Neuropsychiatric Interview the development and validation of a structured diagnostic psychiatric interview for DSM-IV and ICD-10. J Clin Psychiatry. 1998; 59 Suppl. 20:22-33; quiz 34-57.
Shen H W, Hagino Y, Kobayashi H, Shinohara-Tanaka K, Ikeda K, Yamamoto H, Yamamoto T, Lesch K P, Murphy D L, Hall F S, Uhl G R, Sora I (2004) Regional differences in extracellular dopamine and serotonin assessed by in vivo microdialysis in mice lacking dopamine and/or serotonin transporters. Neuropsychopharmacology 29:1790-9.
Skolnick M, Popik P, Janowsky A, Beer B, Lippa A: Antidepressant-like actions of DOV 21, 947: A triple reuptake inhibitor. European Journal of Pharmacology 2003 461:99-104
Trivedi M H, Fava M, Wisniewski S R, Thase M E, Quitkin F, Warden D, Ritz L, Nierenberg A A, Lebowitz B D, Biggs M M, Luther J F, Shores-Wilson K, Rush A J; STAR*D Study Team. Medication augmentation after the failure of SSRIs for depression. N Engl J Med. 2006 Mar. 23; 354(12):1243-52
Vizi E S, Zsilla G, Caron M G, Kiss J P (2004) Uptake and release of norepinephrine by serotonergic terminals in norepinephrine transporter knock-out mice: implications for the action of selective serotonin reuptake inhibitors. J Neurosci 24:7888-94.
Warren, W. L. Revised Hamilton Rating Scale for Depression (RHSD). (1994) Los Angeles, Western Psychological Services.
Wright A, Luedtke K Y, and VanDenBerg C (2011) Duloxetine in the treatment of chronic pain due to fibromyalgia and diabetic neuropathy. J. Pain Res. 4:1-10.
Zhou F C, Tao-Cheng J H, Segu L, Patel T, Wang Y (1998) Serotonin transporters are located on the axons beyond the synaptic junctions: anatomical and functional evidence. Brain Res 805:241-54.
Zhou F M, Liang Y, Salas R, Zhang L, De Biasi M, Dani J (2005) Corelease of Dopamine and Serotonin from Striatal Dopamine Terminals. Neuron 46:65-74.

What is claimed is:

1. A method for treating or preventing a central nervous system (CNS) disorder in a human subject comprising administering to said subject an effective amount of a composition sufficient to inhibit cellular native and promiscuous uptake of biogenic amine neurotransmitters norepinephrine, serotonin, and dopamine.

2. The method of claim 1, wherein the composition comprises a triple reuptake inhibitor compound.

3. The method of claim 1, wherein said composition inhibits cellular uptake of two, or three, of said biogenic amine neurotransmitters non-uniformly by inhibiting uptake of at least one of said norepinephrine, serotonin and/or dopamine in said subject by a factor of two- to fifteen-fold greater than a potency of said composition for inhibiting uptake of at least one different member of said biogenic amine neurotransmitters.

4. The method of claim 1, wherein said composition inhibits cellular uptake of two, or three, of said biogenic amine neurotransmitters non-uniformly by inhibiting uptake of at least one of said norepinephrine, serotonin and/or dopamine in said subject by a factor of two- to ten-fold greater than a potency of said composition for inhibiting uptake of at least one different member of said biogenic amine neurotransmitters.

5. The method of claim 1, wherein the CNS disorder is depression.

6. The method of claim 5, wherein the composition comprises (+)-1-(3,4-dichlorophenyl)-3-azabicyclo[3.1.0]hexane or a pharmaceutically acceptable active salt, polymorph, solvate, hydrate, or prodrug thereof.

7. The method of claim 6, wherein the effective amount is effective to decrease depressive symptoms.

8. The method of claim 7, wherein the effective amount is effective to decrease the human's score on a Montgomery Åsberg Depression Rating Scale to less than or equal to 12.

9. The method of claim 7, wherein the effective amount is effective to decrease the human's score on a Hamilton Rating Scale for Depression to less than or equal to 7.

10. The method of claim 7, wherein the effective amount is effective to decrease the human's score on a Clinical Global Impressions-Improvement score to less than or equal to 2.

11. The method of claim 1, wherein the central nervous system disorder is treatment resistant depression.

12. The method of claim 1, wherein the CNS disorder is an attention deficit disorder.

13. The method of claim 12, wherein the attention deficit disorder is ADHD.

14. The method of claim 12, wherein the composition comprises (1R,5S)-(+)-1-(naphthalen-2-yl)-3-azabicyclo[3.1.0]hexane or a pharmaceutically acceptable active salt, polymorph, solvate, hydrate, or prodrug thereof.

15. The method of claim 1, wherein the CNS disorder is an anxiety disorder.

16. The method of claim 1, wherein the CNS disorder is obesity.

17. The method of claim 1, wherein the CNS disorder is substance abuse.

18. The method of claim 1, wherein the CNS disorder is a cognitive disorder.

19. The method of claim 1, wherein the CNS disorder is chronic pain state.

20. The method of claim 19, wherein the chronic pain state is selected from the group consisting of neuropathic pain, fibromyalgia, traumatic brain injury, and irritable bowel syndrome.

21. The method of claim 1, wherein the composition further comprises an additional psychotherapeutic agent or drug.

22. The method of claim 21, wherein the additional psychotherapeutic agent is an antidepressant, antipsychotic, anticonvulsant, anxiolytic, stimulant, medication for Parkinson's disease, medication for ADHD, opioid, antiaddictive, or appetite suppressant drug.

23. The method of claim 22, wherein the anti-depressant agent is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

24. The method of claim 19, wherein the additional psychotherapeutic agent is a medication for Parkinson's disease.

25. The method of claim 24, wherein the additional psychotherapeutic agent is L-DOPA.

26. A method for increasing endogenous levels of one or more monoamine neurotransmitter levels in a mammalian subject comprising administering to said subject having a central nervous system (CNS) disease or condition amenable to treatment involving increasing levels of one or more monoamine neurotransmitters, an effective amount of composition sufficient to inhibit cellular native and promiscuous uptake of biogenic amine neurotransmitters norepinephrine, serotonin, and dopamine.

27. The method of claim 26, wherein the composition comprises a triple reuptake inhibitor compound.
